(19) United States
(12) Patent Application Publication
(10) Pub. No.: US 2025/0216680 A1
(43) Pub. Date: Jul. 3, 2025

(54) LIGHT GUIDE DEVICE, DISPLAY DEVICE, AND DISPLAY SYSTEM

(71) Applicant: SONY GROUP CORPORATION, TOKYO (JP)

(72) Inventor: MASANORI IWASAKI, TOKYO (JP)

(21) Appl. No.: 18/843,905

(22) PCT Filed: Jan. 20, 2023
(86) PCT No.: PCT/JP2023/001682

§ 371 Date: Sep. 4, 2024

(30) Foreign Application Priority Data
Mar. 15, 2022 (JP) 2022-040360

(51) Int. Cl. G02B 27/01; A61F 2/16; G02B 27/09; G02B 27/42; G02C 7/04

(52) U.S. Cl. CPC G02B 27/0172; A61F 2/1654; G02B 27/0944; G02B 27/4272; G02C 7/049; G02B 2027/013; G02B 2027/0174; G02C 2202/20

(57) ABSTRACT

Provided is a light guide device that can suppress darkening and invisibleness at the center of a display range. A light guide device according to the present technique includes a light guide system that guides light emitted from a light source device to eyeballs, wherein the light guide system has an optical element including first and second diffraction elements that are opposed to each other, light emitted from the light source device and projected to the first diffraction element at a predetermined incident angle is reflected and diffracted at the first diffraction element, the light reflected and diffracted at the first diffraction element is reflected and diffracted at the second diffraction element, the light that is reflected and diffracted at the second diffraction element and is transmitted through the first diffraction element is guided to the eyeballs, the second diffraction element has a lens function, and the first and second diffraction elements vary in refractive index difference and thickness.

### TECHNICAL FIELD

The present technique relates to a light guide device, a display device, and a display system.

### BACKGROUND ART

In recent years, techniques of superimposing images on an outside scene have received attention. Such a technique is also called augmented reality (AR) technique. A head-mounted display is an example of a product using such a technique. A head-mounted display is used while being mounted on the head of a user. In an image display method using a head-mounted display, for example, light from the head-mounted display reaches the eyes of a user in addition to extraneous light, so that the user recognizes that an image of light from the display is superimposed on an image of the outside.

For example, PTL 1 proposes a light guide device including a virtual image optical system that improves a contrast, light use efficiency, and see-through efficiency.

### CITATION LIST

### Patent Literature

### [PTL 1]

JP 2005-148655 A

### SUMMARY

### Technical Problem

However, in the technique proposed in PTL 1, the center of a display range may become dark or invisible.

Thus, the present technique has been devised under such circumstances. A primary object of the present technique is to provide a light guide device that can suppress darkening and invisibleness at the center of a display range.

### Solution to Problem

The present technique provides a light guide device including a light guide system that guides light emitted from a light source device to eyeballs, wherein the light guide system has an optical element including first and second diffraction elements that are opposed to each other, light emitted from the light source device and projected to the first diffraction element at a predetermined incident angle is reflected and diffracted at the first diffraction element, the light transmitted through the first diffraction element is reflected and diffracted at the second diffraction element, and the light transmitted through the first diffraction element via the second diffraction element is guided to the eyeballs, the second diffraction element has a lens function, and the first and second diffraction elements vary in refractive index difference and/or thickness.

The first and second diffraction elements may vary in the product of a refractive index difference and a thickness.

A first product as the product of the refractive index difference and the thickness of the first diffraction element may be larger than a second product as the product of the refractive index difference and the thickness of the second diffraction element.

The first and second diffraction elements may have different refractive index differences.

The first diffraction element may have a larger refractive index difference than the second diffraction element.

The first and second diffraction elements may have different thicknesses.

The second diffraction element may have a larger thickness than the first diffraction element.

The first and second diffraction elements may vary in refractive index difference and thickness.

The first diffraction element may have a larger refractive index difference than the second diffraction element, and the second diffraction element may have a larger thickness than the first diffraction element.

The first and second diffraction elements may be identical in thickness, and the first diffraction element may have a larger refractive index difference than the second diffraction element.

The first and second diffraction elements may be identical in refractive index difference, and the second diffraction element may have a larger thickness than the first diffraction element.

When the first and second diffraction elements have thicknesses in μm, the first product may be 0.2 or more, and the first diffraction element may have a thickness of 1 μm to 100 μm.

When the first and second diffraction elements have thicknesses in μm, the first product may be 0.4 or more, and the first diffraction element may have a thickness of 2 μm to 30 μm.

When the first and second diffraction elements have thicknesses in μm, the second product may be 0.1 or less, and the second diffraction element may have a thickness of 1 μm to 100 μm.

When the first and second diffraction elements have thicknesses in μm, the second product may be 0.02 or less, and the second diffraction element may have a thickness of 10 μm to 40 μm.

The optical axis of the second diffraction element and the normal line of the first diffraction element may be parallel to each other.

The optical axis of the second diffraction element and the normal line of the first diffraction element may be non-parallel.

Light projected to the first diffraction element at the predetermine incident angle may be light that is emitted from the light source device and is transmitted through the second diffraction element.

The light guide system may further include a light guide plate that is at least partially disposed between the first and second diffraction elements and guides light from the light source device, and light projected to the first diffraction element at the predetermine incident angle may be light that is emitted from the light source device and is guided by the light guide plate.

The first diffraction element may reflect and diffract, in a regular reflection direction, light projected to the first diffraction element at the predetermine incident angle.

The first diffraction element may reflect and diffract, in an incident direction, light projected to the first diffraction element at the predetermine incident angle. The first diffraction element may reflect and diffract light projected to the first diffraction element at the predetermine incident angle, in a direction different from both of the regular reflection direction and the incident direction.

At least one of the first and second diffraction elements may have a curved element surface.

At least one of the first and second diffraction elements may be a hologram optical element.

The optical element may be attached to or embedded into the eyeball.

The present technique provides a display device including: the light guide device, and the light source device.

In the display device, the light source device and the light guide device may be integrally provided.

The present technique provides a display system including: the display device, and a controller that controls the display device.

### BRIEF DESCRIPTION OF DRAWINGS

FIG. 1 is an explanatory drawing illustrating that display light is projected to an optical element and the display light is allowed to reach a retina by the optical element.

FIG. 2 is an explanatory drawing of a path before display light incident on the optical element is emitted from the optical element.

FIG. 3 shows the coordinates of diffraction efficiency of display light reaching a retina.

FIG. 4 is an explanatory drawing of a path before display light incident on the optical element is emitted from the optical element.

FIG. 5 is a graph showing the relationship between an incident angle and diffraction efficiency when Δn=0.02 is obtained in an HOE.

FIG. 6 is a graph showing the relationship between an incident angle and diffraction efficiency when Δn=0.04 is obtained in the HOE.

FIG. 7 is a graph showing the relationship between an incident angle and diffraction efficiency when Δn=0.1 is obtained in the HOE.

FIG. 8 is a graph showing the relationship between an incident angle and diffraction efficiency when Δn=0.2 is obtained in the HOE.

FIG. 9 is a graph showing the relationship between an incident angle and diffraction efficiency of two cases where a corresponding incident angle range in the HOE is identical.

FIG. 10 is a graph showing, for a plurality of combinations of Δn and T, the relationship between an incident angle and diffraction efficiency when Δn×T=1.0 is obtained in the HOE.

FIG. 11 is a graph showing, for a plurality of combinations of Δn and T, the relationship between an incident angle and diffraction efficiency when Δn×T=0.8 is obtained in the HOE.

FIG. 12 is a graph showing, for a plurality of combinations of Δn and T, the relationship between an incident angle and diffraction efficiency when Δn×T=0.6 is obtained in the HOE.

FIG. 13 is a graph showing, for a plurality of combinations of Δn and T, the relationship between an incident angle and diffraction efficiency when Δn×T=0.4 is obtained in the HOE.

FIG. 14 is a graph showing, for a plurality of combinations of Δn and T, the relationship between an incident angle and diffraction efficiency when Δn×T=0.2 is obtained in the HOE.

FIG. 15 is a graph showing, for plurality of Δn, the relationship between an incident angle and diffraction efficiency when T=10 μm is obtained in the HOE.

FIG. 16 is an explanatory drawing of function example 1 of the HOE.

FIG. 17 is an explanatory drawing of function example 2 of the HOE.

FIG. 18 is an explanatory drawing of function example 3 of the HOE.

FIG. 19 is an explanatory drawing of function example 4 of the HOE.

FIG. 20 is an explanatory drawing of function example 5 of the HOE.

FIG. 21 illustrates a configuration example of a light guide device according to a first embodiment of the present technique.

FIG. 22 illustrates a configuration example of a light guide device according to a second embodiment of the present technique.

FIG. 23 illustrates a configuration example of a light guide device according to a third embodiment of the present technique.

FIG. 24 illustrates a configuration example of a light guide device according to a fourth embodiment of the present technique.

FIG. 25 illustrates a configuration example of a light guide device according to a fifth embodiment of the present technique.

FIG. 26 illustrates a configuration example of a light guide device according to a sixth embodiment of the present technique.

FIG. 27 illustrates a configuration example of a light guide device according to a seventh embodiment of the present technique.

FIG. 28 illustrates a configuration example of a light guide device according to an eighth embodiment of the present technique.

FIG. 29 illustrates a configuration example of a light guide device according to a ninth embodiment of the present technique.

FIG. 30 illustrates a configuration example of a light guide device according to a tenth embodiment of the present technique.

FIG. 31 illustrates a configuration example of a light guide device according to an eleventh embodiment of the present technique.

FIG. 32 is a functional block diagram illustrating a display system according to a thirteenth embodiment of the present technique.

FIGS. 33A to 33F show diffraction efficiency distributions on an eyeball, for different combinations of the refractive index difference and the film thickness when the product of the refractive index difference and the film thickness of the deflection HOE is constant.

FIGS. 34A to 34F show diffraction efficiency distributions on an eyeball, for different combinations of the refractive index difference and the film thickness when the product of the refractive index difference and the film thickness of the deflection HOE is constant.

### DESCRIPTION OF EMBODIMENTS

Preferred embodiments for implementing the present technique will be described below. The embodiments which will be described below show an example of a representative embodiment of the present technique, and the scope of the present technique should not be narrowly interpreted on the basis of the embodiments. In the drawings, unless otherwise specified, “up” means the upper direction or the upper side in the drawing, “down” means the lower direction or the lower side in the drawing, “left” means the left direction or the left side in the drawing, and “right” means the right direction or the right side in the drawing. The same reference numerals will be applied to the same or equivalent elements or members in the drawings, and repeated descriptions are omitted unless the circumstances are exceptional.

Description will be made in the following order.
1. Outline of Present Technique
2. Detail of Present Technique
3. Light Guide Device of First Embodiment
4. Light Guide Device of Second Embodiment
5. Light Guide Device of Third Embodiment
6. Light Guide Device of Fourth Embodiment
7. Light Guide Device of Fifth Embodiment
8. Light Guide Device of Sixth Embodiment
9. Light Guide Device of Seventh Embodiment
10. Light Guide Device of Eighth Embodiment
11. Light Guide Device of Ninth Embodiment
12. Light Guide Device of Tenth Embodiment
13. Light Guide Device of Eleventh Embodiment
14. Display Device of Twelfth Embodiment
15. Display System of Thirteenth Embodiment

### 1. Outline of Present Technique

First, the outline of the present technique will be described. The present technique relates to a light guide device, a display device, and a display system.

For example, if an AR (Augmented Reality) display device has optical elements including first and second diffraction elements opposed to each other, light reflected and diffracted at the first diffraction element enters the second diffraction element. The light reflected and diffracted at the second diffraction element enters the first diffraction element again. The incident light passes through the first diffraction element and enters eye balls.

However, when the light diffracted at the second diffraction element forms an angle close to the diffraction angle of the first diffraction element, the light incident on the first diffraction element is diffracted again. This reduces light entering eyeballs and thus may cause reduction of light or invisibleness at the center of a display field of view. The detail will be described below.

For example, a display device projects display light onto a contact-lens optical element from an image projection device and forms an image of the display light on retinas through the optical element. The contact-lens optical element includes a reflection volume hologram of a deflection HOE ((Holographic Optical Element), the same applies hereinafter) and a lens HOE (Holographic Optical Element), the same applies hereinafter). The HOE having a structure that diffracts incident light at any angle has a diffraction effect for a design wavelength only at a specific angle.

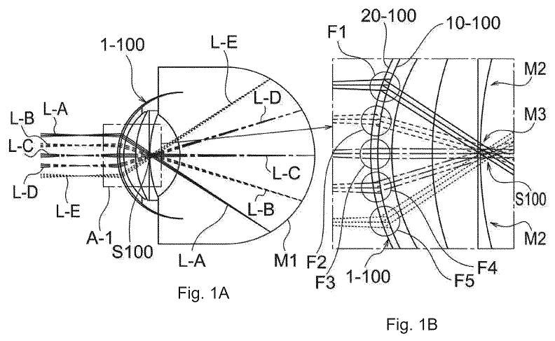

The structure will be described with reference to FIG. 1. FIG. 1 is an explanatory drawing illustrating that five display light beams (L-A to L-E) are projected to a contact-lens optical element 1-100 and the optical element 1-100 causes the display light beams to reach a retina M1 through the optical element 1-100. FIG. 1B is an enlarged view of a part denoted by reference numeral A-1 in FIG. 1A. The five display light beams (L-A to L-E) converge to a point (focus S-100) around a retina M3. Reference numeral M2 denotes an iris. As illustrated in FIG. 1B, the optical element 1-100 is configured with a deflection HOE 10-100 serving as a first diffraction element and a lens HOE 20-100 (a diffraction element having a lens function) serving as a second diffraction element.

As indicated by reference numerals F1 to F5 of FIG. 1B, when the deflection HOE 10-100 has an incident angle and a diffraction angle of 0°, the incident angle of the lens HOE 20-100 and the optical axis of a condenser lens are both set at 0°.

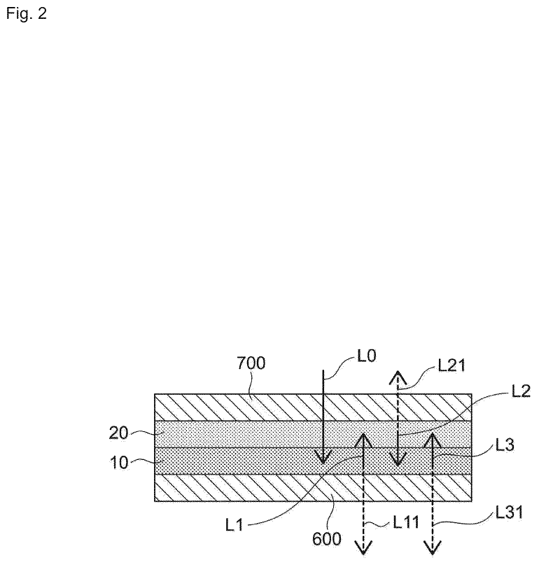

The structure will be described with reference to FIG. 2. FIG. 2 is an explanatory drawing of a path before a display light beam projected to the optical element is emitted from the optical element. The optical element illustrated in FIG. 2 includes a protective layer 700, a lens HOE 20, a deflection HOE 10, and a protective layer 600 in this order from the incident side of incident light L0 (sequentially from the upper side of FIG. 2).

FIG. 2 only illustrates an optical axis component of diffracted light of the lens HOE 20. The incident light L0 passes through the lens HOE 20, a component (light beam L1) enters the deflection HOE 10 and is diffracted, and a component (light beam L11) passes through the deflection HOE 10. In this structure, light not diffracted passes through the HOE unless the diffraction efficiency is 100%.

In this case, as illustrated in FIG. 2, the light beam (light beam L1) is diffracted through the deflection HOE 10, a light beam (light beam L2) is diffracted through the lens HOE 20, and only a transmitted light beam L31 of the light beam L2 having reentered the deflection HOE 10 serves as an image display light beam.

However, in FIG. 1, a light beam enters the lens HOE 20-100, and the optical axis having been condensed, reflected, and diffracted as a lens and the incident angle of the deflection HOE 10-100 for reentry agree with each other. Thus, a light beam component near the optical axis is diffracted through the deflection HOE (for example, a light beam L3 in FIG. 2). A condensing light beam component remote from the optical axis deviates from an angle where the deflection HOE has a diffraction effect, so that the light beam passes through the deflection HOE (for example, a light beam L31 in FIG. 2).

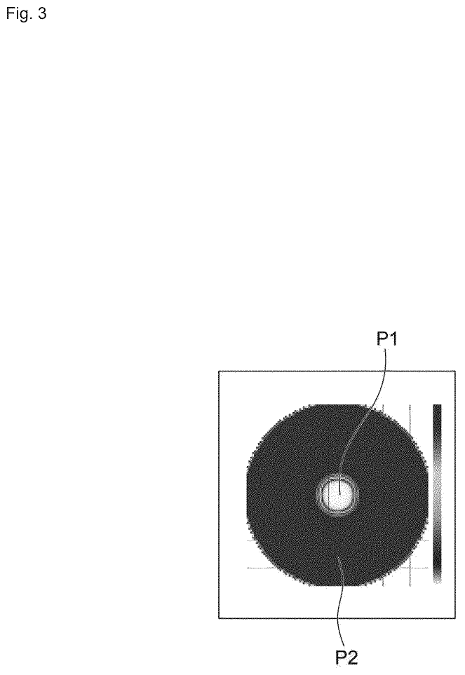

A description will be made with reference to FIG. 3. FIG. 3 shows the coordinates of diffraction efficiency of display light reaching a retina on a contact-lens optical element as a technical example. Reference numeral P2 in FIG. 3 indicates a range of high diffraction efficiency, and reference numeral P1 indicates a range not to be diffracted. In this case, the optical axis of a lens HOE corresponds to a fovea centralis on the retina.

In reality, the diffraction efficiency of 100% cannot be obtained, allowing the passage of part of light near the optical axis of the lens HOE. Only a few of display light beams near the optical axis reach the retina, thereby darkening the display of the central portion of a display range.

Since the display of the fovea centralis, which is the most important in the display range, is darkened, the present technique is devised to avoid this problem.

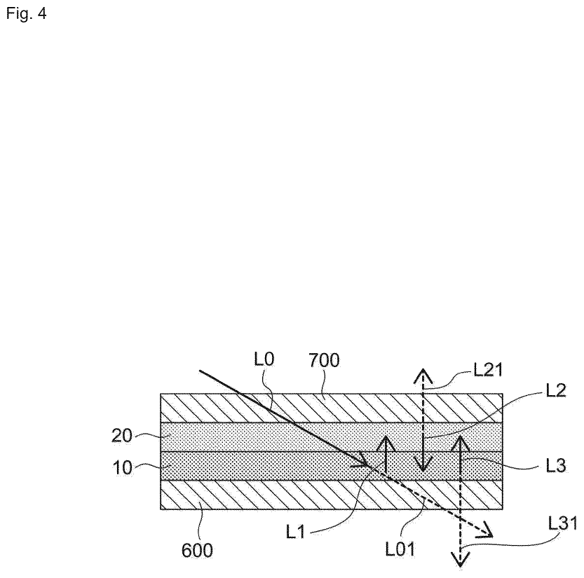

A description will be made with reference to FIG. 4. FIG. 4 is an explanatory drawing of a path before a display light beam projected to the optical element is emitted from the optical element.

A light beam reaching the retina as a display light beam (image display light beam) is only a component having passed through the deflection HOE 10 from among light beams having reentered the deflection HOE 10 (light beam L31). As a schematic path before the image display light beam reaches the retina, (1) the projection of the incident light L0, (2) diffraction through the deflection HOE 10 (light beam light beam L1), (3) diffraction through the lens HOE 20 (light beam L2), (4) passage through the deflection HOE (light beam L31), and (5) arrival at the retina are included in this order.

### 2. Detail of Present Technique

The detail of the present technique will be described below.

### Idea of Inventor

The inventor found that darkening and invisibleness at the center of a display range can be suppressed by adjusting a refractive index difference Δn and/or a film thickness T (thickness) of each of a deflection HOE serving as a first diffraction element and a lens HOE serving as a second diffraction element, the diffraction elements being provided for a contact lens or eyeball-embedded optical element.

Specifically, the inventor focused attention on the fact that the diffraction characteristics of the HOE, that is, diffraction efficiency and a corresponding incident angle range can be changed by changing the refractive index difference Δn and/or a film thickness T (thickness) of the HOE. In this case, “corresponding incident angle range” means the range of the incident angle of light to the HOE when the diffraction efficiency of the HOE exceeds a predetermined value (for example, a predetermined value of 1 to 100%). “Corresponding incident angle range” may be called “effective incident angle range.”

### (Basic Configuration of Light Guide Device)

A light guide device according to the present technique includes a light guide system that guides light emitted from a light source device to eyeballs. The light guide system has an optical element including first and second diffraction elements (for example, a deflection HOE and a lens HOE) that are opposed to each other. Light emitted from a light source device and projected to the first diffraction element (for example, the deflection HOE) at a predetermined incident angle is reflected and diffracted at the first diffraction element, the light reflected and diffracted at the first diffraction element is reflected and diffracted at the second diffraction element (for example, the lens HOE), and the light that is reflected and diffracted at the second diffraction element and is transmitted through the first diffraction element is guided to the eyeballs.

### (Diffraction Characteristics of HOE)

In the present technique, diffraction characteristics will be described in consideration of a concept in which a product Δn×T of a refractive index difference Δn and a film thickness T serves as an index. In this case, the diffraction element is allowed to have a diffraction function by alternately placing two or more kinds of different refractive index regions. A difference in refractive index will be referred to as a refractive index difference.

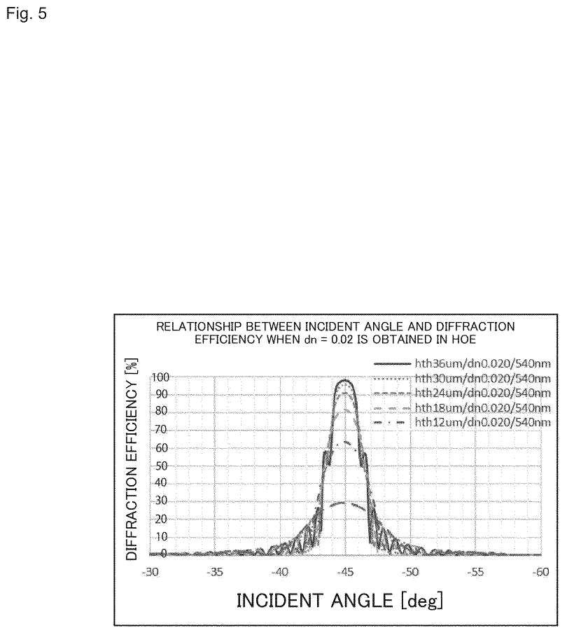

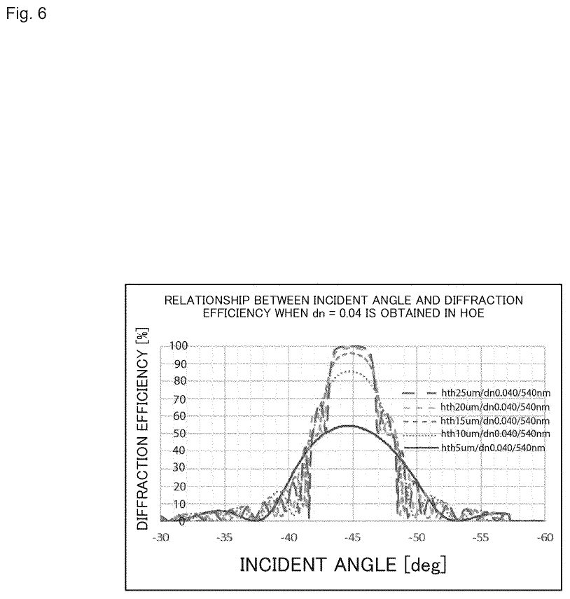

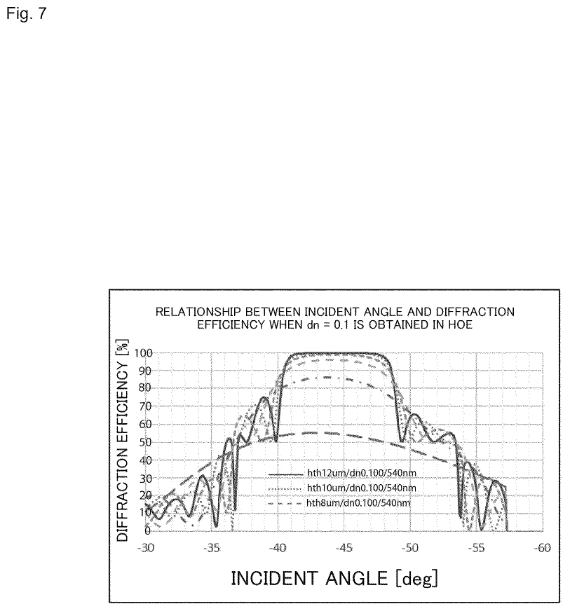

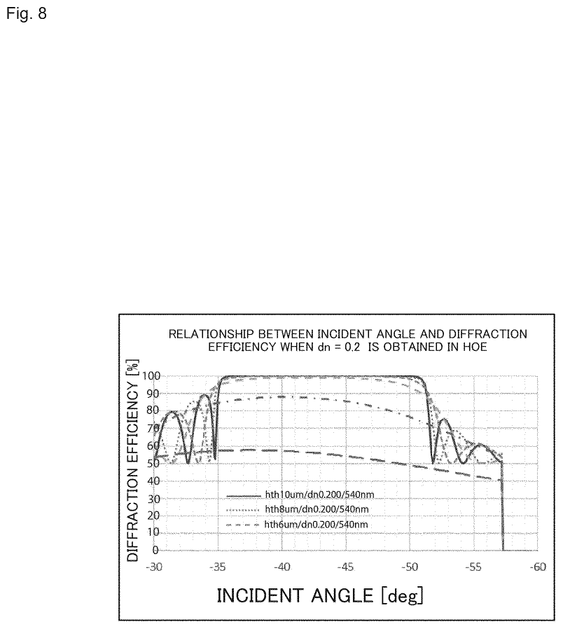

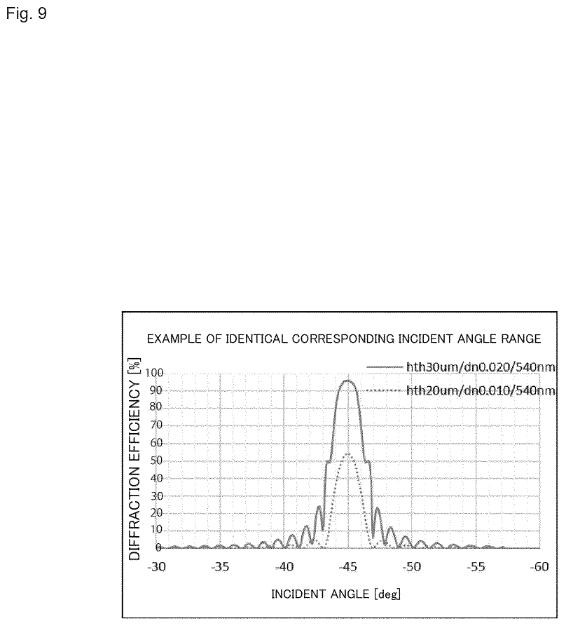

FIG. 5 is a graph showing, for multiple thicknesses T(hth), the relationship between an incident angle and diffraction efficiency when Δn(dn)=0.02 is obtained in the HOE. FIG. 6 is a graph showing, for multiple thicknesses T(hth), the relationship between an incident angle and diffraction efficiency when Δn(dn)=0.04 is obtained in the HOE. FIG. 7 is a graph showing, for multiple thicknesses T(hth), the relationship between an incident angle and diffraction efficiency when Δn(dn)=0.1 is obtained in the HOE. FIG. 8 is a graph showing, for multiple thicknesses T(hth), the relationship between an incident angle and diffraction efficiency when Δn(dn)=0.2 is obtained in the HOE. FIG. 9 is a graph showing the relationship between an incident angle and diffraction efficiency of two cases where a corresponding incident angle range in the HOE is identical. In FIGS. 5 to 9, the wavelength of light incident on the HOE is, for example, 540 nm.

As is evident from FIGS. 5 to 8, when Δn is kept constant, the corresponding incident angle range narrows and the diffraction efficiency increases as T increases, whereas the corresponding incident angle range expands and the diffraction efficiency decreases as T decreases.

A comparison among FIGS. 5 to 8 proves that when a film thickness is kept constant, the corresponding incident angle range tends to expand as Δn increases.

As is evident from FIG. 9, when the corresponding incident angle range is identical, fewer wavelengths are disturbed in the corresponding incident angle range and display is less affected as Δn×T decreases.

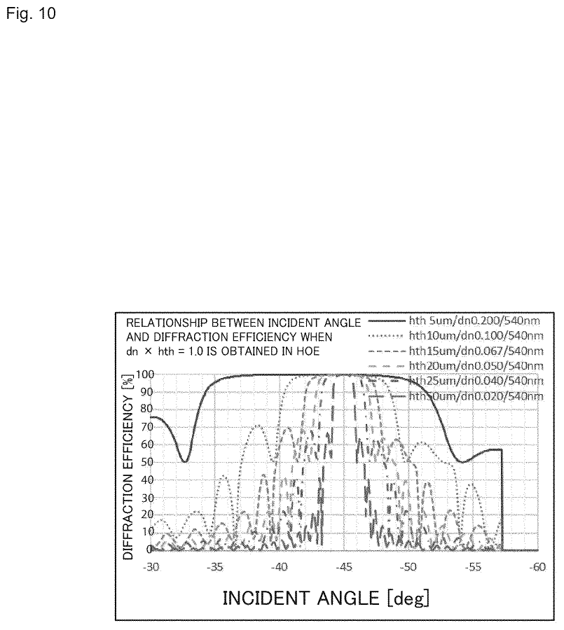

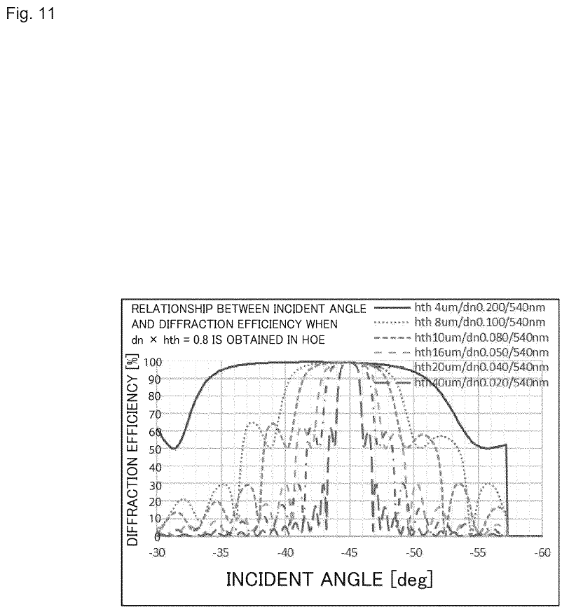

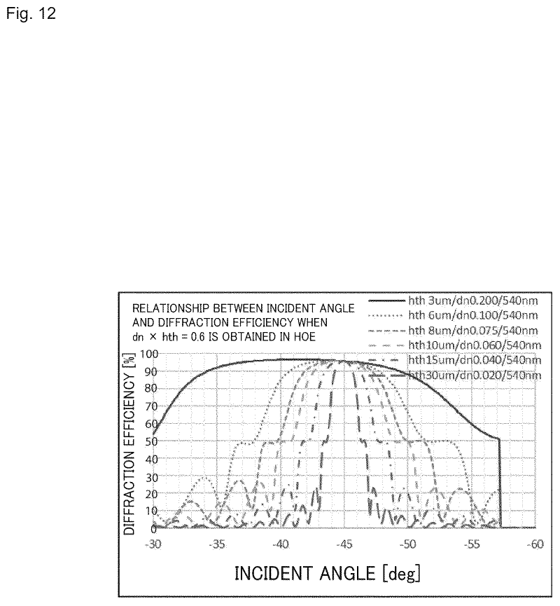

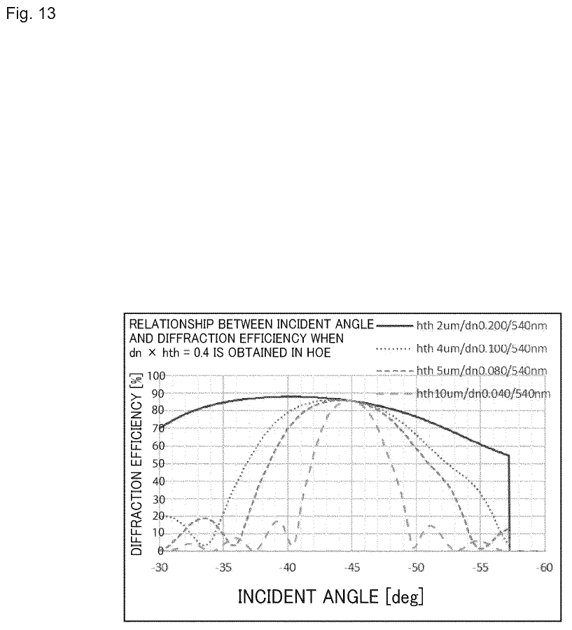

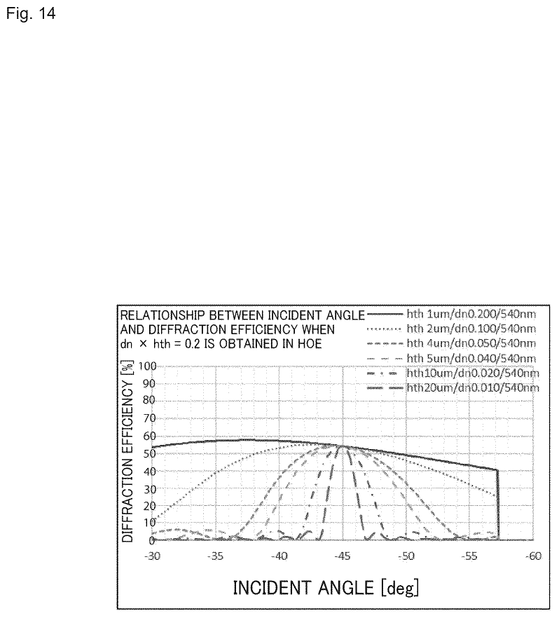

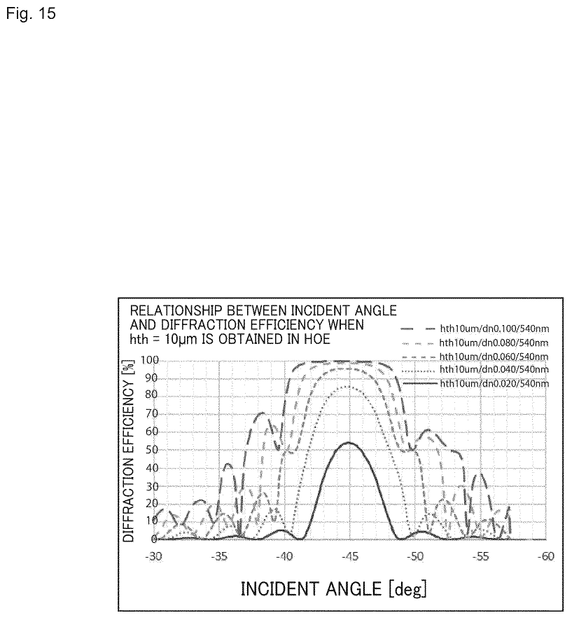

FIG. 10 is a graph showing, for multiple combinations of Δn(dn) and T(hth), the relationship between an incident angle and diffraction efficiency when Δn×T=1.0 is obtained in the HOE. FIG. 11 is a graph showing, for multiple combinations of Δn(dn) and T(hth), the relationship between an incident angle and diffraction efficiency when Δn×T=0.8 is obtained in the HOE. FIG. 12 is a graph showing, for multiple combinations of Δn(dn) and T(hth), the relationship between an incident angle and diffraction efficiency when Δn×T=0.6 is obtained in the HOE. FIG. 13 is a graph showing, for multiple combinations of Δn(dn) and T(hth), the relationship between an incident angle and diffraction efficiency when Δn×T=0.4 is obtained in the HOE. FIG. 14 is a graph showing, for multiple combinations of Δn(dn) and T(hth), the relationship between an incident angle and diffraction efficiency when Δn×T=0.2 is obtained in the HOE. FIG. 15 is a graph showing, for multiple Δn(dn), the relationship between an incident angle and diffraction efficiency when T=10 μm is obtained in the HOE. In FIGS. 10 to 15, the wavelength of light incident on the HOE is, for example, 540 nm.

As is evident from FIGS. 10 to 14, a corresponding incident angle range can be more stably obtained as Δn×T increases. However, when Δn×T exceeds 0.8 μm, the peak of diffraction efficiency (maximum diffraction efficiency) is saturated, leading to difficulty in obtaining the effect of improvement. The smaller the film thickness, the larger the corresponding incident angle range. In the case of a film thickness on the order of μm, the manufacturing tolerance is large and variations in characteristics increase when the film thickness is 5 μm or less.

As is evident from FIGS. 10 to 14, the diffraction efficiency of the HOE is proportionate to Δn×T.

As is evident from FIG. 15, when T is kept constant, the corresponding incident angle range expands and the diffraction efficiency improves as Δn increases.

As is evident from the examination, the diffraction characteristics of the HOE can be controlled by adjusting Δn and/or T. The inventor successfully suppressed darkening and invisibleness at the center of a display range by providing diffraction characteristics suitable for the deflection HOE and the lens HOE according to this method.

Specifically, the deflection HOE and the lens HOE preferably vary in refractive index difference Δn and/or film thickness T (thickness). The deflection HOE and the lens HOE preferably vary in the product Δn×T of the refractive index difference Δn and film thickness T (thickness). In other words, for a first product Δn1×T1 of a refractive index difference Δn1 and a film thickness T1 of the deflection HOE and a second product Δn2×T2 of a refractive index difference Δn2 and a film thickness T2 of the lens HOE, Δn1×T1>Δn2×T2 or Δn1×T1<Δn2×T2 is preferably established.

Since eyeballs rotate in the configuration for projection from the image projection device to the contact-lens optical element, the deflection HOE preferably has a wide corresponding incident angle range and high diffraction efficiency. In other words, when Δn1×T1 is kept constant, the deflection HOE preferably has a large value of Δn1 and a small value of T1. This is because light incident on the deflection HOE varies in incident angle and low diffraction efficiency increases zeroth-order light that may cause an adverse effect.

In the following example, light projected from the image projection device and caused to enter the deflection HOE in a direction of, for example, 60° from the normal line of the contact-lens optical element is reflected and diffracted at, for example, 60° in a regular reflection direction. The optical axis of the lens HOE and the normal line of the deflection HOE are parallel to each other.

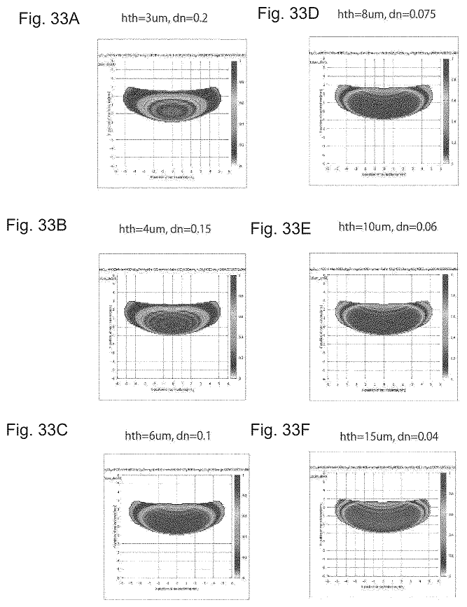

FIGS. 33A to 33F show diffraction efficiency distributions on an eyeball, for different combinations of the refractive index difference and the film thickness when the product of the refractive index difference and the film thickness of the deflection HOE is constant (same). In FIGS. 33A to 33F, the horizontal axis indicates a position in Y-axis direction on the light guide device and the vertical axis indicates a position in X-axis direction on the light guide device. In FIGS. 33A to 33F, a darker region has higher diffraction efficiency, whereas a lighter region has lower diffraction efficiency. In the examples of FIGS. 33A to 33F, the film thickness of the lens HOE is set at 3 μm and the refractive index difference thereof is set at 0.2.

FIG. 33A shows the diffraction efficiency distribution of a combination of a film thickness hth=3 μm and dn=0.2. FIG. 33B shows the diffraction efficiency distribution of a combination of a film thickness hth=4 μm and dn=0.15. FIG. 33C shows the diffraction efficiency distribution of a combination of a film thickness hth=6 μm and dn=0.1. FIG. 33D shows the diffraction efficiency distribution of a combination of a film thickness hth=8 μm and dn=0.075. FIG. 33E shows the diffraction efficiency distribution of a combination of a film thickness hth=10 μm and dn=0.06. FIG. 33F shows the diffraction efficiency distribution of a combination of a film thickness hth=15 μm and dn=0.04.

As is evident from FIGS. 33A to 33F, when the first product Δn1×T1 of the refractive index difference Δn1 and the film thickness T1 is kept constant, the deflection HOE has a larger fovea centralis, which has high diffraction efficiency on the retina, as Δn1 decreases (as T1 increases). Thus, the deflection HOE preferably has relatively small Δn1 (relatively large T1) when Δn1×T1 is kept constant.

The lens HOE preferably has a narrow corresponding incident angle range. This is because less diffraction is more preferable when light passes through the lens HOE. The lens HOE does not necessarily need to have high diffraction efficiency because low diffraction efficiency only reduces light use efficiency and does not affect display. In other words, the lens HOE preferably has a large T value.

In the following example, light projected from the image projection device and caused to enter the deflection HOE in a direction of, for example, 60° from the normal line of the contact-lens optical element is reflected and diffracted at, for example, 80° in a regular reflection direction. The optical axis of the lens HOE and the normal line of the deflection HOE are parallel to each other.

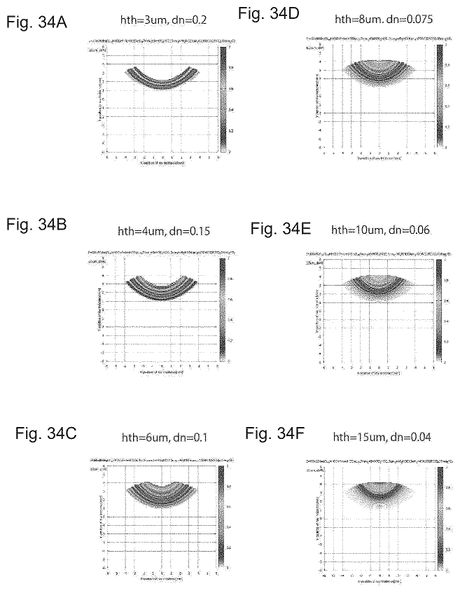

Light from the image projection device first passes through the lens HOE and enters the deflection HOE. If incident light is first diffracted through the lens HOE, a transmitted light beam may be lost. Thus, diffraction efficiency first obtained through the lens HOE is preferably minimized, and a diffraction characteristic distribution in a fovea centralis region in FIGS. 34A to 34F is preferably minimized. FIGS. 34A to 34F show diffraction efficiency distributions on an eyeball, for different combinations of the refractive index difference and the film thickness when the product of the refractive index difference and the film thickness of the deflection HOE for the light that is first incident on the lens HOE is constant (same). In FIGS. 34A to 34F, the horizontal axis indicates a position in Y-axis direction on the light guide device and the vertical axis indicates a position in X-axis direction on the light guide device. In FIGS. 34A to 34F, a darker region has higher diffraction efficiency, whereas a lighter region has lower diffraction efficiency. In the examples of FIGS. 34A to 34F, the film thickness of the lens HOE is set at 15 μm and the refractive index difference thereof is set at 0.04.

FIG. 34A shows the diffraction efficiency distribution of a combination of a film thickness hth=3 μm and dn=0.2. FIG. 34B shows the diffraction efficiency distribution of a combination of a film thickness hth=4 μm and dn=0.15. FIG. 34C shows the diffraction efficiency distribution of a combination of a film thickness hth=6 μm and dn=0.1. FIG. 34D shows the diffraction efficiency distribution of a combination of a film thickness hth=8 μm and dn=0.075. FIG. 34E shows the diffraction efficiency distribution of a combination of a film thickness hth=10 μm and dn=0.06. FIG. 34F shows the diffraction efficiency distribution of a combination of a film thickness hth=15 μm and dn=0.04.

As is evident from FIGS. 34A to 34F, when the second product Δn2×T2 of the refractive index difference Δn2 and the film thickness T2 is kept constant, the lens HOE has a smaller range with high diffraction efficiency in a fovea centralis region on the retina as Δn2 decreases (as T2 increases), the range being generated on the retina through the lens HOE by light first entering the lens HOE. Thus, the lens HOE preferably has relatively small Δn2 (relatively large T2) when Δn2×T2 is kept constant.

The first product Δn1×T1 of the refractive index difference Δn1 and the film thickness T1 of the deflection HOE is preferably larger than the second product Δn2×T2 of the refractive index difference Δn2 and the film thickness T2 of the lens HOE (Δn1×T1>Δn2×T2). Thus, the corresponding incident angle range of the deflection HOE can be relatively large, the corresponding incident angle range of the lens HOE can be relatively small, and the diffraction efficiency of the deflection HOE can be relatively high.

The deflection HOE and the lens HOE may have different refractive index differences. In this case, the deflection HOE preferably has a larger refractive index difference than the lens HOE (Δn1>Δn2). Thus, the diffraction efficiency of the deflection HOE can be relatively high.

The deflection HOE and the lens HOE may have different film thicknesses T (thicknesses). In this case, the lens HOE preferably has a larger thickness than the deflection HOE (T2>T1). Thus, the corresponding incident angle range of the lens HOE can be relatively small.

The deflection HOE and the lens HOE may have different refractive index differences and thicknesses. In this case, the deflection HOE preferably has a larger refractive index difference than the lens HOE (Δn1>Δn2) and the lens HOE preferably has a larger thickness than the deflection HOE (T2>T1). Thus, the diffraction efficiency of the deflection HOE can be relatively high, and the corresponding incident angle range of the lens HOE can be relatively small.

The deflection HOE and the lens HOE may have the same thickness (T1=T2), and the deflection HOE may have a larger refractive index difference than the lens HOE (Δn1>Δn2) (see FIG. 15). Thus, the diffraction efficiency and corresponding incident angle range of the deflection HOE can be relatively high.

The deflection HOE and the lens HOE may have the same refractive index difference (Δn1=Δn2). In this case, although the deflection HOE and the lens HOE only need to have different film thicknesses, the lens HOE preferably has a larger thickness than the deflection HOE. Thus, the corresponding incident angle range of the deflection HOE can be relatively large, and the corresponding incident angle range of the lens HOE can be relatively small.

The first product Δn1×T1 is preferably 0.2 or more, and the deflection HOE preferably has a thickness of 1 μm to 100 μm (see FIGS. 10 to 14). Thus, the corresponding incident angle range of the deflection HOE can be expanded while variations in characteristics are suppressed.

The first product Δn1×T1 is more preferably 0.4 or more, and the deflection HOE more preferably has a thickness of 2 μm to 30 μm (see FIGS. 10 to 14). Thus, the corresponding incident angle range and the diffraction efficiency of the deflection HOE can be increased while variations in characteristics are suppressed.

Δn2 is preferably 0.1 or less, and the lens HOE preferably has a thickness of 1 μm to 100 μm. Thus, the corresponding incident angle range of the lens HOE can be narrowed while variations in characteristics are suppressed.

Δn2 is more preferably 0.05 or less (preferably 0.02 or less), and the lens HOE more preferably has a thickness of 10 μm to 40 μm. Thus, the corresponding incident angle range of the lens HOE can be narrowed while variations in characteristics are suppressed.

If the deflection HOE diffracts incident light in the regular reflection direction, formula (1) below is preferably established for an incident angle θi of light to the deflection HOE, a lens opening diameter D, and a lens focal distance f.

$$θ i \geq a tan ( D / ( 2 f ) ) \qquad ( 1 )$$

If the deflection HOE diffracts incident light in the regular reflection direction, formula (2) below is preferably established for an incident angle θi of light to the deflection HOE, a lens opening diameter D, a lens focal distance f, and a diffraction angle characteristic range width ΔθwiL of the lens HOE.

$$θ i \geq a tan ( D / ( 2 f ) ) + Δ θ wiL \qquad ( 2 )$$

If the deflection HOE diffracts incident light in the regular reflection direction, formula (3) below is preferably established for an incident angle θi of light to the deflection HOE, an opening diameter D, a lens focal distance f, and a diffraction angle characteristic range width ΔθwiH of the deflection HOE.

$$θ i \geq a tan ( D / ( 2 f ) ) + Δ θ wiH \qquad ( 3 )$$

If the deflection HOE diffracts incident light in the incident direction, formula (4) below is preferably established for an incident angle θi of light to the deflection HOE, a deflection diffraction angle θHi of the deflection HOE, a lens focal distance f, and a diffraction angle characteristic range width ΔθwiL of the lens HOE.

$$θ i \geq θ Hi \geq a tan ( D / ( 2 f ) ) \qquad ( 4 )$$

If the deflection HOE diffracts incident light in the incident direction, formula (5) below is more preferably established for an incident angle θi of light to the deflection HOE, a deflection diffraction angle θHi, a lens focal distance f, a diffraction angle characteristic range width ΔθwiL of the lens HOE, and a diffraction angle characteristic range width ΔθwiH of the deflection HOE.

$$θ i \geq θ Hi + Δ θ wiH \geq a tan ( D / ( 2 f ) ) + Δ θ wiL \qquad ( 5 )$$

### Function Example 1 of HOE

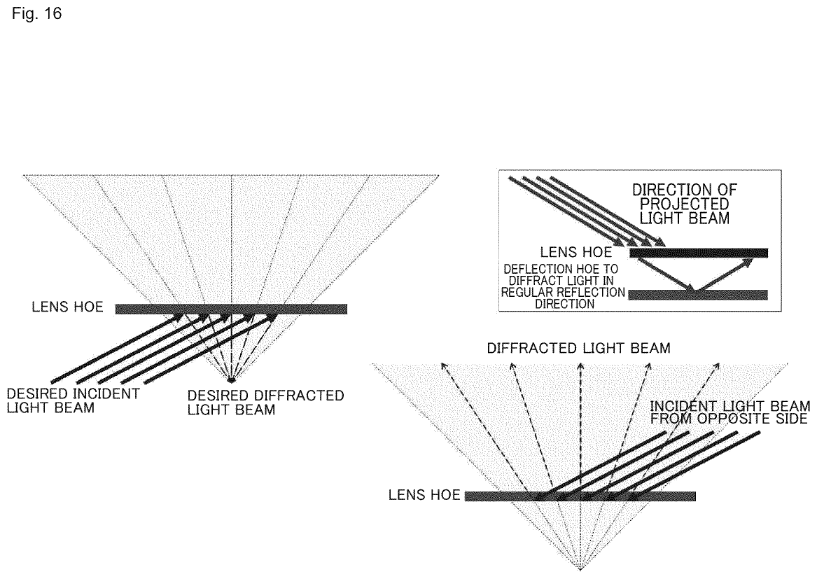

FIG. 16 is an explanatory drawing of function example 1 of the HOE. In this case, as illustrated in the upper right drawing of FIG. 16, a projected light beam transmitted through the lens HOE is diffracted at the deflection HOE in the regular reflection direction. As illustrated in the upper left drawing of FIG. 16, a light beam reflected and diffracted at the deflection HOE in the regular reflection direction is incident as a desired incident light beam on the lens HOE having diffraction efficiency of 100%, and the light beam is reflected and diffracted to converge as a desired diffracted light beam at the lens HOE. As illustrated in the lower right drawing of FIG. 16, a light beam projected to the lens HOE having diffraction efficiency of 1 from the side opposite to the deflection HOE is reflected and diffracted to be dispersed opposite to a light convergence point of a diffracted light beam at the lens HOE.

### Function Example 2 of HOE

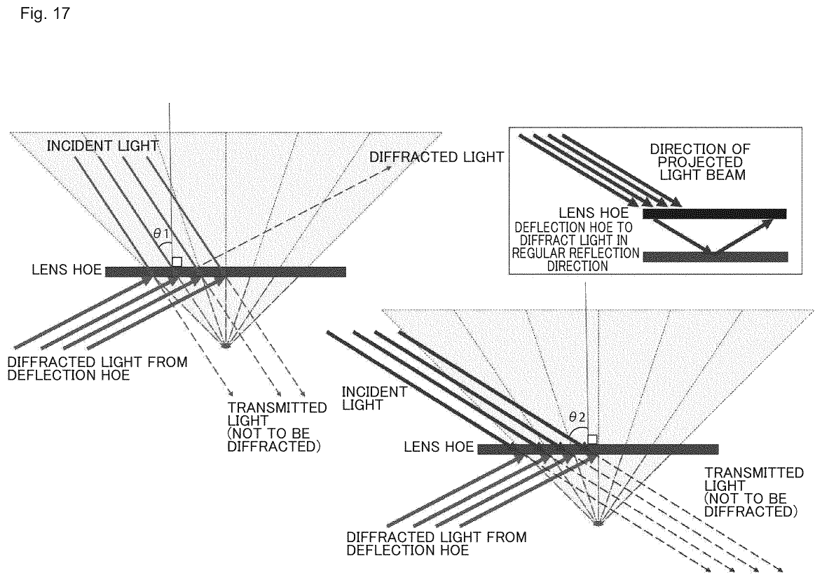

FIG. 17 is an explanatory drawing of function example 2 of the HOE. As illustrated in the upper right drawing of FIG. 17, a projected light beam transmitted through the lens HOE is reflected and diffracted at the deflection HOE in the regular reflection direction. As illustrated in the upper left drawing of FIG. 17, a part of a projected light beam (incident light) to the lens HOE at an incident angle θ1 (an incident angle in the diffraction angle characteristic range width of the deflection HOE and near the diffraction angle characteristic range width of the lens HOE) is transmitted through the lens HOE, is reflected and diffracted at the deflection HOE in the regular reflection direction, and is caused to enter the lens HOE again, and the other part is reflected and diffracted to the outside as diffracted light at the lens HOE. The light beam caused to enter the lens HOE again is reflected and diffracted to converge as diffracted light at the lens HOE. In other words, in the example of the upper left drawing of FIG. 17, incident light is partially lost. In the example of the lower right drawing of FIG. 17, a projected light beam (incident light) to the lens HOE at an incident angle θ2 (an incident angle in the diffraction angle characteristic range width of the deflection HOE and considerably larger than 01 outside the diffraction angle characteristic range width of the lens HOE) is mostly transmitted through the lens HOE, is reflected and diffracted at the deflection HOE in the regular reflection direction, and is caused to enter the lens HOE again. The light beam caused to enter the lens HOE again is reflected and diffracted to be converged as diffracted light at the lens HOE. In other words, in the example of the lower right drawing of FIG. 17, incident light is hardly lost. As described above, the incident angle of a projected light beam (incident light) to the deflection HOE is set at a predetermined value or larger (for example, θi in formulas (1) to (5)), thereby suppressing a loss of incident light.

### Function Example 3 of HOE

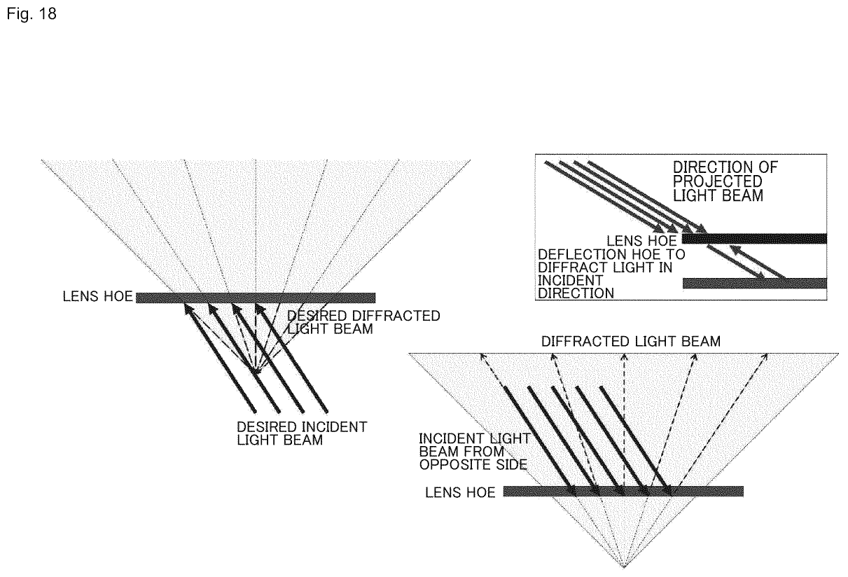

FIG. 18 is an explanatory drawing of function example 4 of the HOE. In this case, as illustrated in the upper right drawing of FIG. 18, a projected light beam transmitted through the lens HOE is reflected and diffracted at the deflection HOE in the incident direction. As illustrated in the upper left drawing of FIG. 18, a light beam reflected and diffracted at the deflection HOE in the incident direction is incident as a desired incident light beam on the lens HOE having diffraction efficiency of 100%, and the light beam is reflected and diffracted to converge as a desired diffracted light beam at the lens HOE. As illustrated in the lower right drawing of FIG. 18, a light beam projected to the lens HOE having diffraction efficiency of 1 from the side opposite to the deflection HOE is reflected and diffracted to be dispersed opposite to a light convergence point of a diffracted light beam at the lens HOE.

### Function Example 4 of HOE

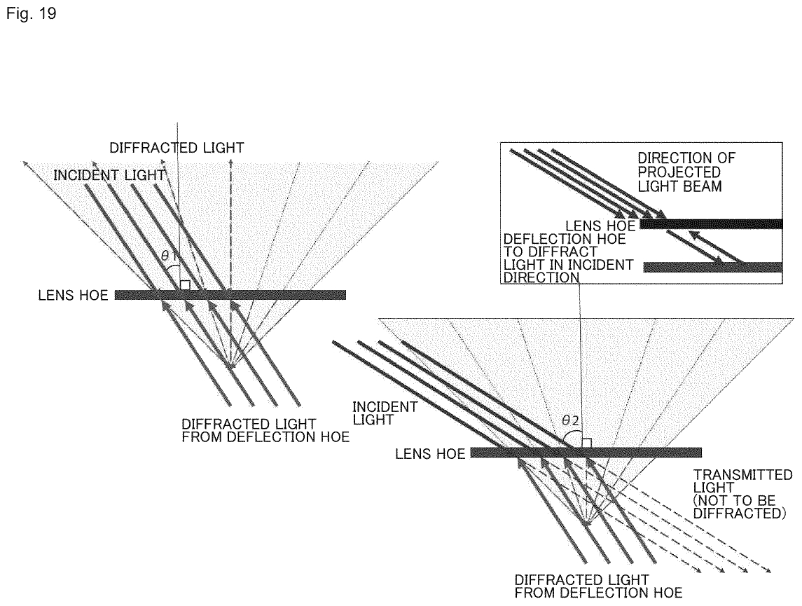

FIG. 19 is an explanatory drawing of function example 2 of the HOE. As illustrated in the upper right drawing of FIG. 19, a projected light beam transmitted through the lens HOE is reflected and diffracted at the deflection HOE in the incident direction. As illustrated in the upper left drawing of FIG. 19, a part of a projected light beam (incident light) to the lens HOE at an incident angle θ1 (an incident angle in the diffraction angle characteristic range width of the deflection HOE and near the diffraction angle characteristic range width of the lens HOE) is transmitted through the lens HOE, is reflected and diffracted at the deflection HOE in the incident direction, and is caused to enter the lens HOE again, and the other part is reflected and diffracted to the outside as diffracted light at the lens HOE. The light beam caused to enter the lens HOE again is reflected and diffracted to converge as diffracted light at the lens HOE. In other words, in the example of the upper left drawing of FIG. 19, incident light is partially lost. In the example of the lower right drawing of FIG. 19, a projected light beam (incident light) to the lens HOE at an incident angle θ2 (an incident angle in the diffraction angle characteristic range width of the deflection HOE and considerably larger than 01 outside the diffraction angle characteristic range width of the lens HOE) is mostly transmitted through the lens HOE, is reflected and diffracted at the deflection HOE in the incident direction, and is caused to enter the lens HOE again. The light beam caused to enter the lens HOE again is reflected and diffracted to be converged as diffracted light at the lens HOE. In other words, in the example of the lower right drawing of FIG. 19, incident light is hardly lost. As described above, the incident angle of a projected light beam (incident light) to the deflection HOE is set at a predetermined value or larger (for example, θi in formulas (1) to (5)), thereby suppressing a loss of incident light.

### Function Example 5 of HOE

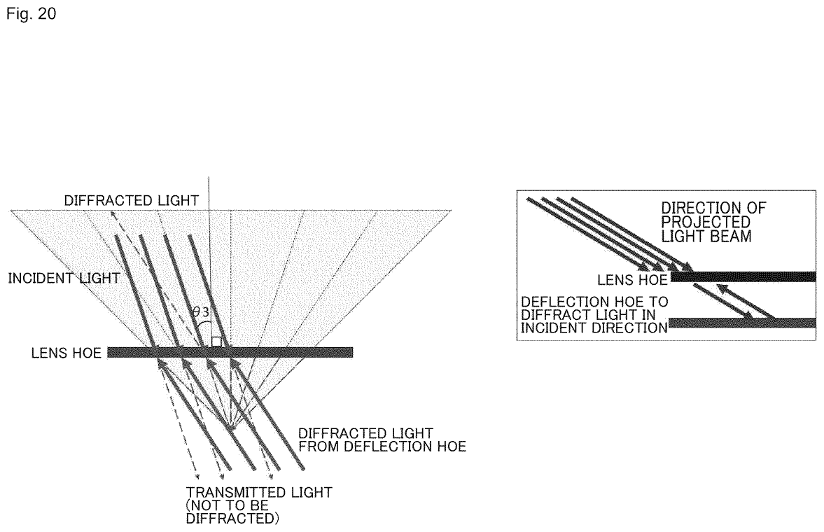

FIG. 20 is an explanatory drawing of function example 5 of the HOE. As illustrated in the right drawing of FIG. 20, a projected light beam transmitted through the lens HOE is reflected and diffracted at the deflection HOE in the incident direction. As illustrated in the left drawing of FIG. 20, a part of a projected light beam (incident light) to the lens HOE at an incident angle θ3 (an incident angle in the diffraction angle characteristic range width of the deflection HOE and smaller than 01 in the diffraction angle characteristic range width of the lens HOE) is transmitted through the lens HOE, is reflected and diffracted at the deflection HOE in the incident direction, and is caused to enter the lens HOE again, and the other part (equivalent) is reflected and diffracted to the outside as diffracted light at the lens HOE. The light beam caused to enter the lens HOE again is reflected and diffracted to converge as diffracted light at the lens HOE. In other words, in the example of FIG. 20, incident light is partially lost (larger than that in the example of the upper left drawing of FIG. 19).

### (Configuration, Operation, and Effect of Light Guide Device, Display Device, and Display System)

The light guide device according to the present technique may be provided with, for example, a light guide system including a contact-lens or eyeball-embedded optical element. The contact-lens optical element is attached to the surface of an eyeball (for example, a cornea). The eyeball-embedded optical element is embedded in an eyeball (for example, a crystalline lens). The display device according to the present technique may be provided with the light guide device and the image projection device (light source device) that projects image light to the contact-lens or eyeball-embedded optical element of the light guide system of the light guide device. The display device may be a head-mounted type. The image projection device may be configured with, for example, a light source and a projection optical system. In the present technique, a substrate (for example, a light guide plate) can be also used as in the display device of a fifth embodiment, which will be described later, according to the present technique. A configuration for implementing the light guide device according to the present technique is not to be limited to a specific configuration.

In the present technique, light (light beam) is projected from the image projection device (light source device) to the second diffraction element (for example, the lens HOE). The light projected to the second diffraction element passes through the second diffraction element (for example, the lens HOE) as in, for example, the light guide device of a sixth embodiment or the light guide device of a seventh embodiment, which will be described later, according to the present technique, and then the light enters the first diffraction element at a different angle from the optical axis of the lens function of the second diffraction element. The incident light beam is reflected and diffracted by a periodic structure, which is provided in the first diffraction element, in any direction different from that of the optical axis of the lens function of the second diffraction element. The reflected and diffracted light enters the second diffraction element.

The incident light beam can be reflected and diffracted by the periodic structure provided in the second diffraction element, in a direction that condenses (or disperses) light with the lens optical axis provided almost vertically with respect to a contact lens surface by the lens function of the second diffraction element.

The optical axis component of light reflected and diffracted at the second diffraction element enters the first diffraction element again. However, the reflection diffraction action of the periodic structure provided in the first diffraction element does not occur, so that the optical axis component of light reflected and diffracted at the second diffraction element is transmitted without being diffracted. The transmitted light passes through an eyeball and is incident on a retina.

In the light incident on the eyeball, light on the lens optical axis of the second diffraction element reaches the fovea centralis of the eyeball. Thus, the amount of light does not decrease in an image at a central portion of a displayed image, enabling display at the fovea centralis.

The optical axis of the second diffraction element (for example, the lens HOE) and the normal line of the first diffraction element (deflection HOE) may be parallel to each other.

The optical axis of the second diffraction element (for example, the lens HOE) and the normal line of the first diffraction element (deflection HOE) may be non-parallel.

Light projected to the first diffraction element (for example, the deflection HOE) at a predetermine incident angle may be light that is emitted from the light source device and is transmitted through the second diffraction element (for example, the lens HOE).

The light guide system further includes a light guide plate that is at least partially disposed between the first and second diffraction elements (for example, the deflection HOE and the lens HOE) and guides light from the light source device. Light projected to the first diffraction element (for example, the deflection HOE) at a predetermine incident angle may be light that is emitted from the light source device and is guided by the light guide plate.

The first diffraction element (for example, the deflection HOE) may reflect and diffract, in the regular reflection direction, light projected to the first diffraction element at a predetermine incident angle.

The first diffraction element (for example, the deflection HOE) may reflect and diffract, in the incident direction, light projected to the first diffraction element at a predetermine incident angle.

The first diffraction element (for example, the deflection HOE) may reflect and diffract light projected to the first diffraction element at a predetermine incident angle, in a direction different from both of the regular reflection direction and the incident direction.

At least one of the first and second diffraction elements (for example, the deflection HOE and the lens HOE) may have a curved element surface.

The light source device and the light guide device may be integrally provided. For example, a self-emitting display element may be mounted in the optical element including the first and second diffraction elements.

The display device according to the present technique can be configured with the light guide device according to the present technique and the light source device that projects light into the light guide device.

The display device according to the present technique can be configured with the display device according to the present technique and a controller that controls the display device.

Hereinafter, preferred embodiments for implementing the present technique will be described in detail with reference to the drawings. In the following embodiments, examples of representative embodiments of the present technique will be described, and the scope of the present technique should not be narrowly interpreted on the basis of the embodiments.

### 3. Light Guide Device of First Embodiment of Present Technique

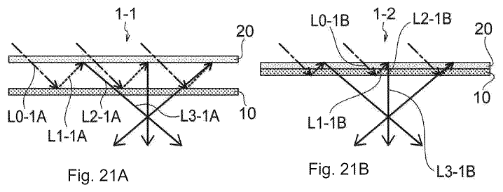

Referring to FIG. 21, a light guide device according to a first embodiment of the present technique will be described below.

FIG. 21 illustrates a configuration example of the light guide device of the first embodiment according to the present technique. Specifically, FIG. 21A illustrates a light guide device 1-1, and FIG. 21B illustrates a light guide device 1-2.

### (Light Guide Device 1-1)

The light guide device 1-1 includes at least an optical element configured with a first diffraction element 10 and a second diffraction element 20 that are opposed to each other. In the light guide device 1-1, an image is formed on the basis of image display light (display light) L3-1A that passes through the first diffraction element 10 and is emitted out of the light guide device 1-1. In the light guide device 1-1, the first diffraction element 10 and the second diffraction element 20 may be disposed close to each other.

Each of the first diffraction element 10 and the second diffraction element 20 may be any one of a volume hologram optical element, a diffraction grating optical element, and a metasurface optical element.

The first diffraction element 10 has a deflection function of deflecting incident light L0-1A to the entry side of the incident light L0-1A on the first diffraction element 10, the incident light L0-1A being projected with a predetermined wavelength to the first diffraction element 10 by the second diffraction element 20 at a predetermined incident angle deviated from the optical axis of a lens. The first diffraction element 10 emits first diffracted light L1-1A deflected by the deflection function.

As illustrated in FIG. 21A, in the three-dimensional coordinate system of the light guide device 1-1, the normal line of the first diffraction element 10 extends from the origin point of the three-dimensional coordinate system in +z axis direction (upward direction in FIG. 21A), the top surface (the upper surface in FIG. 21A) of the first diffraction element 10 is an x-y plane (a plane extending from the near side to the remote side in the drawing), the incident light L0-1A on the first diffraction element 10 is light directed to the origin point of the three-dimensional coordinate system from any point in +z axis direction (upward direction in FIG. 21A), in the range of the second quadrant of an x-y coordinate system (a coordinate system directed from the near side to the remote side in the drawing), and in the range of the third quadrant or on −x axis (a leftward axis in the drawing). The first diffracted light L1-1A emitted from the first diffraction element 10 is light directed from the origin point of the three-dimensional coordinate system to any point in +z axis direction (upper direction in FIG. 21A), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on −y axis (an axis from the remote side to the near side in the drawing). The light is deflected in the regular reflection direction with respect to the incident light L0-1A.

The second diffraction element 20 has a lens function for the first diffracted light L1-1A that is projected with a predetermined wavelength at a predetermined incident angle to the second diffraction element 20. The lens function means a function of condensing (convex lens) or dispersing (concave lens) light (light beam) (the same applies hereinafter about a lens function).

The second diffraction element 20 has a deflection function of deflecting the first diffracted light L1-1A to the entry side of the first diffracted light L1-1A on the second diffraction element 20, the first diffracted light L1-1A being projected with a predetermined wavelength at a predetermined incident angle to the second diffraction element 20. The second diffraction element 20 emits second diffracted light L2-1A deflected by the deflection function. The second diffracted light L2-1A is condensed by the lens function and enters the first diffraction element 10 again. At this point, the first diffraction element 10 does not have the effect of deflecting light in the optical axis direction of the lens. Thus, the second diffracted light L2-1A passes though the first diffraction element 10 and reaches a retina as image display light L3-1A, thereby preventing darkening and invisibleness at the center of a display field of view, the center corresponding to the lens optical axis.

Furthermore, in the light guide device 1-1, the direction of the incident angle of the incident light L0-1A and the direction of the diffraction angle of the first diffracted light L1-1A are different from the optical axis direction of the second diffraction element 20.

### (Light Guide Device 1-2)

The light guide device 1-2 includes at least an optical element configured with a first diffraction element 10 and a second diffraction element 20 that are opposed to each other. In the light guide device 1-2, an image is formed on the basis of image display light (display light) L3-1B that passes through the first diffraction element 10 and is emitted out of the light guide device 1-2. As illustrated in FIG. 21B, in the light guide device 1-2, the first diffraction element 10 and the second diffraction element 20 are disposed in contact.

The first diffraction element 10 has a deflection function of deflecting incident light L0-1B to the entry side of the incident light L0-1B on the first diffraction element 10, the incident light L0-1B being projected with a predetermined wavelength to the first diffraction element 10 by the second diffraction element 20 at a predetermined incident angle deviated from the optical axis of a lens function. The first diffraction element 10 emits first diffracted light L1-1B deflected by the deflection function.

As illustrated in FIG. 21B, in the three-dimensional coordinate system of the light guide device 1-2, the normal line of the first diffraction element 10 extends from the origin point of the three-dimensional coordinate system in +z axis direction (upward direction in FIG. 21B), the top surface (the upper surface in FIG. 21B) of the first diffraction element 10 is an x-y plane (a plane extending from the near side to the remote side in the drawing), the incident light L0-1B on the first diffraction element 10 is light directed to the origin point of the three-dimensional coordinate system from any point in +z axis direction (upward direction in FIG. 21B), in the range of the second quadrant of an x-y coordinate system (a coordinate system directed from the near side to the remote side in the drawing), and in the range of the third quadrant or on −x axis (a leftward axis in the drawing). The first diffracted light L1-1B emitted from the first diffraction element 10 is light directed from the origin point of the three-dimensional coordinate system to any point in +z axis direction, in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on y axis (an axis from the remote side to the near side in the drawing). The light is deflected in the regular reflection direction with respect to the incident light L0-1B.

The second diffraction element 20 has a lens function for the first diffracted light L1-1B that is projected with a predetermined wavelength at a predetermined incident angle to the second diffraction element 20.

The second diffraction element 20 has a deflection function of deflecting the first diffracted light L1-1B to the entry side of the first diffracted light L1-1B on the second diffraction element 20, the first diffracted light L1-1B being projected with a predetermined wavelength at a predetermined incident angle to the second diffraction element 20. The second diffraction element 20 emits second diffracted light L2-1B deflected by the deflection function.

In the light guide device 1-2, the direction of the incident angle of the incident light L0-1B and the direction of the diffraction angle of the first diffracted light L1-1B are different from the optical axis direction of the second diffraction element 20.

The contents of the light guide device according to the first embodiment of the present technique can be applied to after-mentioned light guide devices according to second to eleventh embodiments of the present technique unless any technical contradictions arise.

### 4. Light Guide Device of Second Embodiment of Present Technique

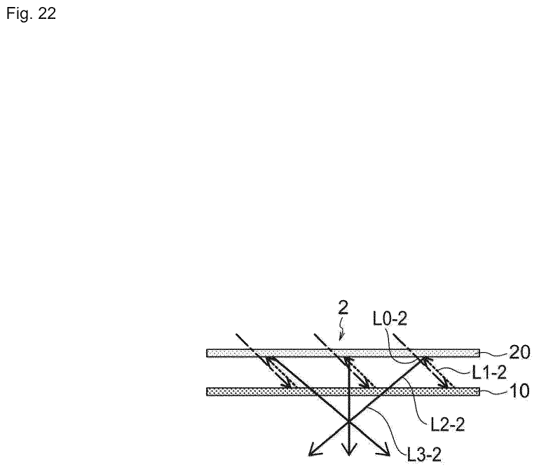

Referring to FIG. 22, a light guide device according to a second embodiment of the present technique will be described below. In the light guide device according to the second embodiment of the present technique, a first diffraction element has the effect of deflecting light in an incident direction and reflecting and diffracting the light after the light is projected to the first diffraction element by a second diffraction element at an angle deviated from the optical axis of a lens. Examples of the first diffraction element and the second diffraction element include a volume HOE.

FIG. 22 illustrates a configuration example of the light guide device according to the second embodiment of the present technique. Specifically, FIG. 22 illustrates a light guide device 2.

### (Light Guide Device 2)

The light guide device 2 includes at least an optical element configured with a first diffraction element 10 and a second diffraction element 20 that are opposed to each other. In the light guide device 2, an image is formed on the basis of image display light (display light) L3-2 that passes through the first diffraction element 10 and is emitted out of the light guide device 2. In the light guide device 2, the first diffraction element 10 and the second diffraction element 20 may be disposed close to each other. The first diffraction element 10 and the second diffraction element 20 may be disposed in contact, which is not illustrated in FIG. 22.

The first diffraction element 10 has a deflection function of deflecting incident light L0-2 to the entry side of the incident light L0-2 on the first diffraction element 10, the incident light L0-2 being projected with a predetermined wavelength to the first diffraction element 10 by the second diffraction element 20 at a predetermined incident angle deviated from the optical axis of a lens. The first diffraction element 10 emits first diffracted light L1-2 deflected by the deflection function.

As illustrated in FIG. 22, in the three-dimensional coordinate system of the light guide device 2, the normal line of the first diffraction element 10 extends from the origin point of the three-dimensional coordinate system in +z axis direction (upward direction in FIG. 22), the top surface (the upper surface in FIG. 22) of the first diffraction element 10 is an x-y plane (a plane extending from the near side to the remote side in the drawing), the incident light L0-2 on the first diffraction element 10 is light directed to the origin point of the three-dimensional coordinate system from any point in +z axis direction (upward direction in FIG. 22), in the range of the second quadrant of an x-y coordinate system (a coordinate system directed from the near side to the remote side in the drawing), and in the range of the third quadrant or on −x axis (a leftward axis in the drawing). The first diffracted light L1-2 emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in the incident direction with respect to the incident light L0-2.

The second diffraction element 20 has a lens function for the first diffracted light L1-2 that is projected with a predetermined wavelength at a predetermined incident angle to the second diffraction element 20.

The second diffraction element 20 has a deflection function of deflecting the first diffracted light L1-2 to the entry side of the first diffracted light L1-2 on the second diffraction element 20, the first diffracted light L1-2 being projected with a predetermined wavelength to the second diffraction element 20. The second diffraction element 20 emits second diffracted light L2-2 deflected by the deflection function.

Furthermore, in the light guide device 2, the direction of the incident angle of the incident light L0-2 and the direction of the diffraction angle of the first diffracted light L1-2 are different from the optical axis direction of the second diffraction element 20.

The contents of the light guide device according to the second embodiment of the present technique can be applied to the light guide device according to the first embodiment of the present technique and after-mentioned light guide devices according to third to eleventh embodiments of the present technique unless any technical contradictions arise.

### 5. Light Guide Device of Third Embodiment of Present Technique

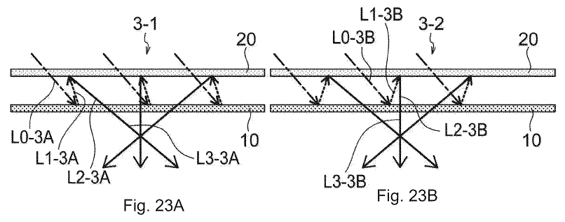

Referring to FIG. 23, a light guide device according to a third embodiment of the present technique will be described below. A feature of the light guide device according to the third embodiment of the present technique is that a light beam entering a first diffraction element forms an angle different from the angle of a light beam entering a second diffraction element and the angle of a light beam diffracted by the second diffraction element. Thus, in the case of a configuration where a light beam passes though the second diffraction element and enters the first diffraction element, the light guide device does not have the effect of condensing (convex lens effect) or dispersing (concave lens effect) a light beam incident on the second diffraction element and reflecting and diffracting the light beam. This allows the passage of the light beam incident on the second diffraction element.

FIG. 23 illustrates a configuration example of the light guide device according to the third embodiment of the present technique. Specifically, FIG. 23A illustrates a light guide device 3-1, and FIG. 23B illustrates a light guide device 3-2.

### (Light Guide Device 3-1)

The light guide device 3-1 includes at least an optical element configured with a first diffraction element 10 and a second diffraction element 20 that are opposed to each other. In the light guide device 3-1, an image is formed on the basis of image display light (display light) L3-3A that passes through the first diffraction element 10 and is emitted out of the light guide device 2. In the light guide device 3-1, the first diffraction element 10 and the second diffraction element 20 may be disposed close to each other. The first diffraction element 10 and the second diffraction element 20 may be disposed in contact, which is not illustrated in FIG. 23A.

The first diffraction element 10 has a deflection function of deflecting incident light L0-3A to the entry side of the incident light L0-3A on the first diffraction element 10, the incident light L0-3A being projected with a predetermined wavelength at a predetermined incident angle to the first diffraction element 10. The first diffraction element 10 emits first diffracted light L1-3A deflected by the deflection function.

As illustrated in FIG. 23A, in the three-dimensional coordinate system of the light guide device 3-1, the normal line of the first diffraction element 10 extends from the origin point of the three-dimensional coordinate system in +z axis direction (upward direction in FIG. 23A), the top surface (the upper surface in FIG. 23A) of the first diffraction element 10 is an x-y plane (a plane extending from the near side to the remote side in the drawing), the incident light L0-3A on the first diffraction element 10 is light directed to the origin point of the three-dimensional coordinate system from any point in +z axis direction (upward direction in FIG. 23A), in the range of the second quadrant of an x-y coordinate system (a coordinate system directed from the near side to the remote side in the drawing), and in the range of the third quadrant or on −x axis (a leftward axis in the drawing). The first diffracted light L1-3A emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in a direction different from the regular reflection direction and the incident direction with respect to the incident light L0-3A.

The second diffraction element 20 has a lens function for the first diffracted light L1-3A that is projected with a predetermined wavelength at a predetermined incident angle to the second diffraction element 20.

The second diffraction element 20 has a deflection function of deflecting the first diffracted light L1-1B to the entry side of the first diffracted light L1-3A on the second diffraction element 20, the first diffracted light L1-3A being projected with a predetermined wavelength at a predetermined incident angle to the second diffraction element 20. The second diffraction element 20 emits second diffracted light L2-3A deflected by the deflection function.

Furthermore, in the light guide device 3-1, the direction of the incident angle of the incident light L0-3A and the direction of the diffraction angle of the first diffracted light L1-3A are different from the optical axis direction of the second diffraction element 20.

### (Light Guide Device 3-2)

The light guide device 3-2 includes at least an optical element configured with a first diffraction element 10 and a second diffraction element 20 that are opposed to each other. In the light guide device 3-2, an image is formed on the basis of image display light (display light) L3-3B that passes through the first diffraction element 10 and is emitted out of the light guide device 3-2. In the light guide device 3-2, the first diffraction element 10 and the second diffraction element 20 may be disposed close to each other. The first diffraction element 10 and the second diffraction element 20 may be disposed in contact, which is not illustrated in FIG. 23B.

The first diffraction element 10 has a deflection function of deflecting incident light L0-3B to the entry side of the incident light L0-3B on the first diffraction element 10, the incident light L0-3B being projected with a predetermined wavelength at a predetermined incident angle to the first diffraction element 10. The first diffraction element 10 emits first diffracted light L1-3B deflected by the deflection function.

As illustrated in FIG. 23B, in the three-dimensional coordinate system of the light guide device 3-2, the normal line of the first diffraction element 10 extends from the origin point of the three-dimensional coordinate system in +z axis direction (upward direction in FIG. 23B), the top surface (the upper surface in FIG. 23B) of the first diffraction element 10 is an x-y plane (a plane extending from the near side to the remote side in the drawing), the incident light L0-3B on the first diffraction element 10 is light directed to the origin point of the three-dimensional coordinate system from any point in +z axis direction (upward direction in FIG. 23B), in the range of the second quadrant of an x-y coordinate system (a coordinate system directed from the near side to the remote side in the drawing), and in the range of the third quadrant or on −x axis (a leftward axis in the drawing). The first diffracted light L1-3B emitted from the first diffraction element 10 is light directed from the origin point of the three-dimensional coordinate system to any point in +z axis direction (upward direction in FIG. 23B), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on −y axis (an axis from the remote side to the near side in the drawing). The light is deflected in a direction different from both of the incident direction and the regular reflection direction with respect to the incident light L0-3B.

The second diffraction element 20 has a lens function for the first diffracted light L1-3B that is projected with a predetermined wavelength at a predetermined incident angle to the second diffraction element 20.

The second diffraction element 20 has a deflection function of deflecting the first diffracted light L1-3B to the entry side of the first diffracted light L1-3B on the second diffraction element 20, the first diffracted light L1-3B being projected with a predetermined wavelength at a predetermined incident angle to the second diffraction element 20. The second diffraction element 20 emits second diffracted light L2-3B deflected by the deflection function.

Furthermore, in the light guide device 3-2, the direction of the incident angle of the incident light L0-3B and the direction of the diffraction angle of the first diffracted light L1-3B are different from the optical axis direction of the second diffraction element 20.

The contents of the light guide device according to the third embodiment of the present technique can be applied to the light guide devices according to the first and second embodiments of the present technique and after-mentioned light guide devices according to fourth to eleventh embodiments of the present technique unless any technical contradictions arise.

### 6. Light Guide Device of Fourth Embodiment of Present Technique

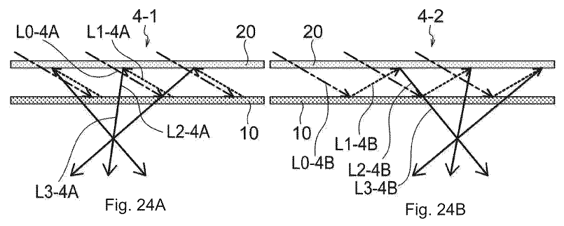

Referring to FIG. 24, a light guide device according to a fourth embodiment of the present technique will be described below. In the light guide device according to the fourth embodiment of the present technique, a feature is that the lens optical axis of a second diffraction element tilts from a vertical line with respect to the second diffraction element.

FIG. 24 illustrates a configuration example of the light guide device according to the fourth embodiment of the present technique. Specifically, FIG. 24A illustrates a light guide device 4-1, and FIG. 24B illustrates a light guide device 4-2.

### (Light Guide Device 4-1)

The light guide device 4-1 includes at least an optical element configured with a first diffraction element 10 and a second diffraction element 20 that are opposed to each other. In the light guide device 4-1, an image is formed on the basis of image display light (display light) L3-4A that passes through the first diffraction element 10 and is emitted out of the light guide device 4-1. In the light guide device 4-1, the first diffraction element 10 and the second diffraction element 20 may be disposed close to each other. The first diffraction element 10 and the second diffraction element 20 may be disposed in contact, which is not illustrated in FIG. 24A.

The first diffraction element 10 has a deflection function of deflecting incident light L0-4A to the entry side of the incident light L0-4A on the first diffraction element 10, the incident light L0-4A being projected with a predetermined wavelength at a predetermined incident angle to the first diffraction element 10. The first diffraction element 10 emits first diffracted light L1-4A deflected by the deflection function.

As illustrated in FIG. 24A, in the three-dimensional coordinate system of the light guide device 4-1, the normal line of the first diffraction element 10 extends from the origin point of the three-dimensional coordinate system in +z axis direction (upward direction in FIG. 24A), the top surface (the upper surface in FIG. 24A) of the first diffraction element 10 is an x-y plane (a plane extending from the near side to the remote side in the drawing), the incident light L0-4A on the first diffraction element 10 is light directed to the origin point of the three-dimensional coordinate system from any point in +z axis direction (upward direction in FIG. 24A), in the range of the second quadrant of an x-y coordinate system (a coordinate system directed from the near side to the remote side in the drawing), and in the range of the third quadrant or on −x axis (a leftward axis in the drawing). The first diffracted light L1-4A emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in the incident direction with respect to the incident light L0-4A.

The second diffraction element 20 has a lens function for the first diffracted light L1-4A that is projected with a predetermined wavelength at a predetermined incident angle to the second diffraction element 20.

The second diffraction element 20 has a deflection function of deflecting the first diffracted light L1-4A to the entry side of the first diffracted light L1-4A on the second diffraction element 20, the first diffracted light L1-4A being projected with a predetermined wavelength at a predetermined incident angle to the second diffraction element 20. The second diffraction element 20 emits second diffracted light L2-4A deflected by the deflection function.

In the light guide device 4-1, the direction of the incident angle of the incident light L0-4A and the direction of the diffraction angle of the first diffracted light L1-4A are different from the optical axis direction of the second diffraction element 20.

### (Light Guide Device 4-2)

The light guide device 4-2 includes at least an optical element configured with a first diffraction element 10 and a second diffraction element 20 that are opposed to each other. In the light guide device 4-2, an image is formed on the basis of image display light (display light) L3-4B that passes through the first diffraction element 10 and is emitted out of the light guide device 4-2. In the light guide device 4-2, the first diffraction element 10 and the second diffraction element 20 may be disposed close to each other. The first diffraction element 10 and the second diffraction element 20 may be disposed in contact, which is not illustrated in FIG. 24B.

The first diffraction element 10 has a deflection function of deflecting incident light L0-4B to the entry side of the incident light L0-4B on the first diffraction element 10, the incident light L0-4B being projected with a predetermined wavelength at a predetermined incident angle to the first diffraction element 10. The first diffraction element 10 emits first diffracted light L1-4B deflected by the deflection function.

As illustrated in FIG. 24B, in the three-dimensional coordinate system of the light guide device 4-2, the normal line of the first diffraction element 10 extends from the origin point of the three-dimensional coordinate system in +z axis direction (upward direction in FIG. 24B), the top surface (the upper surface in FIG. 24B) of the first diffraction element 10 is an x-y plane (a plane extending from the near side to the remote side in the drawing), the incident light L0-4B on the first diffraction element 10 is light directed to the origin point of the three-dimensional coordinate system from any point in +z axis direction (upward direction in FIG. 24B), in the range of the second quadrant of an x-y coordinate system (a coordinate system directed from the near side to the remote side in the drawing), and in the range of the third quadrant or on −x axis (a leftward axis in the drawing). The first diffracted light L1-4B emitted from the first diffraction element 10 is light directed from the origin point of the three-dimensional coordinate system to any point in +z axis direction (upper direction in FIG. 24B), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on y axis (an axis from the remote side to the near side in the drawing). The light is deflected in the regular reflection direction with respect to the incident light L0-4B.

The second diffraction element 20 has a lens function for the first diffracted light L1-4B that is projected with a predetermined wavelength at a predetermined incident angle to the second diffraction element 20.

The second diffraction element 20 has a deflection function of deflecting the first diffracted light L1-4B to the entry side of the first diffracted light L1-4B on the second diffraction element 20, the first diffracted light L1-4B being projected with a predetermined wavelength at a predetermined incident angle to the second diffraction element 20. The second diffraction element 20 emits second diffracted light L2-4B deflected by the deflection function.

In the light guide device 4-2, the direction of the incident angle of the incident light L0-4B and the direction of the diffraction angle of the first diffracted light L1-4B are different from the optical axis direction of the second diffraction element 20.

The contents of the light guide device according to the fourth embodiment of the present technique can be applied to the light guide devices according to the first to third embodiments of the present technique and after-mentioned light guide devices according to fifth to eleventh embodiments of the present technique unless any technical contradictions arise.

### 7. Light Guide Device of Fifth Embodiment of Present Technique

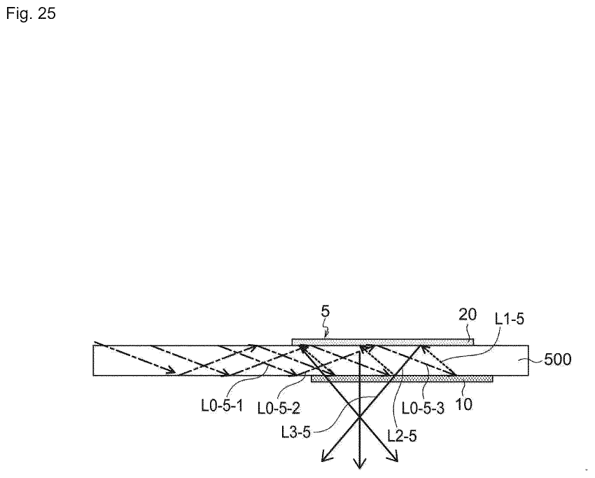

Referring to FIG. 25, a light guide device according to a fifth embodiment of the present technique will be described below. A feature of the light guide device according to the fifth embodiment of the present technique is the provision of a substrate (e.g., a light guide plate or a GRIN lens) in contact with a first diffraction element. For example, the substrate (e.g., a light guide plate or a GRIN lens) may be provided between the first diffraction element and a second diffraction element. The substrate (e.g., a light guide plate or a GRIN lens) guides light incident on the first diffraction element, the guided light is deflected to be reflected and diffracted by a first optical element, and the light is reflected and diffracted through a lens by a second diffraction element.

FIG. 25 illustrates a configuration example of the light guide device according to the fifth embodiment of the present technique. Specifically, FIG. 25 illustrates a light guide device 5.

### (Light Guide Device 5)

The light guide device 5 includes at least an optical element configured with a first diffraction element 10 and a second diffraction element 20 that are opposed to each other. In the light guide device 5, an image is formed on the basis of image display light (display light) L3-5 that passes through the first diffraction element 10 and is emitted out of the light guide device 5.

A light guide plate 500 is provided between the first diffraction element 10 and the second diffraction element 20. In the light guide plate 500, light L0-5-1, light L0-5-2, and light L05-3 are sequentially propagated and while being reflected. The light L05-3 is projected as incident light into the first diffraction element 10.

The first diffraction element 10 has a deflection function of deflecting incident light L0-5-3 to the entry side of the incident light L0-5-3 on the first diffraction element 10, the incident light L0-5-3 being projected with a predetermined wavelength at a predetermined incident angle to the first diffraction element 10. The first diffraction element 10 emits first diffracted light L1-5 deflected by the deflection function.

As illustrated in FIG. 25, in the three-dimensional coordinate system of the light guide device 5, the normal line of the first diffraction element 10 extends from the origin point of the three-dimensional coordinate system in +z axis direction (upward direction in FIG. 25), the top surface (the upper surface in FIG. 25) of the first diffraction element 10 is an x-y plane (a plane extending from the near side to the remote side in the drawing), the incident light L0-5-3 on the first diffraction element 10 is light directed to the origin point of the three-dimensional coordinate system from any point in +z axis direction (upward direction in FIG. 25), in the range of the second quadrant of an x-y coordinate system (a coordinate system directed from the near side to the remote side in the drawing), and in the range of the third quadrant or on −x axis (a leftward axis in the drawing). The first diffracted light L1-5 emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in a direction different from the regular reflection direction and the incident direction with respect to the incident light L0-5-3.

The second diffraction element 20 has a lens function for the first diffracted light L1-5 that is projected with a predetermined wavelength at a predetermined incident angle to the second diffraction element 20.

The second diffraction element 20 has a deflection function of deflecting the first diffracted light L1-5 to the entry side of the first diffracted light L1-5 on the second diffraction element 20, the first diffracted light L1-5 being projected with a predetermined wavelength at a predetermined incident angle to the second diffraction element 20. The second diffraction element 20 emits second diffracted light L2-5 deflected by the deflection function.

In the light guide device 5, the direction of the incident angle of the incident light L0-5-3 and the direction of the diffraction angle of the first diffracted light L1-5 are different from the optical axis direction of the second diffraction element 20.

The contents of the light guide device according to the fifth embodiment of the present technique can be applied to the light guide devices according to the first to fourth embodiments of the present technique and after-mentioned light guide devices according to sixth to eleventh embodiments of the present technique unless any technical contradictions arise.

### 8. Light Guide Device of Sixth Embodiment of Present Technique

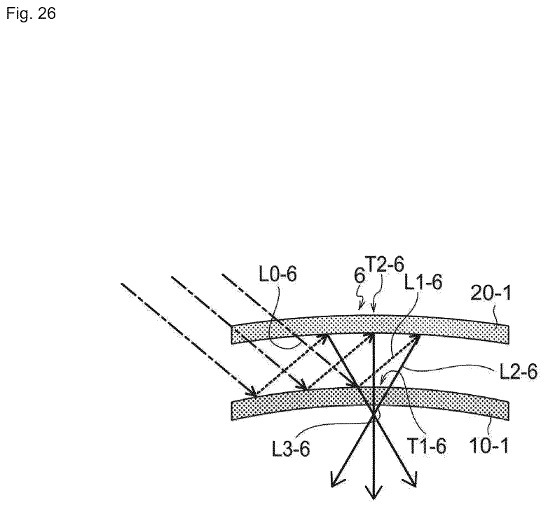

Referring to FIG. 26, a light guide device according to a sixth embodiment of the present technique will be described below.

FIG. 26 illustrates a configuration example of the light guide device according to the sixth embodiment of the present technique. Specifically, FIG. 26 illustrates a light guide device 6.

### (Light Guide Device 6)

The light guide device 6 includes at least an optical element configured with a first diffraction element 10-1 and a second diffraction element 20-1 that are opposed to each other. In the light guide device 6, an image is formed on the basis of image display light (display light) L3-6 that passes through the first diffraction element 10-1 and is emitted out of the light guide device 6. In the light guide device 6, the first diffraction element 10-1 and the second diffraction element 20-1 may be disposed close to each other. The first diffraction element 10-1 and the second diffraction element 20-1 may be disposed in contact, which is not illustrated in FIG. 26.

Each of the first diffraction element 10-1 and the second diffraction element 20-1 may be any one of a volume hologram optical element, a diffraction grating optical element, and a metasurface optical element.

In the light guide device 6, the first diffraction element 10-1 has a curved shape such that an upward protruding shape T1-6 is formed substantially at the central portion of the first diffraction element 10-1, and the second diffraction element 20-1 has a curved shape such that an upward-protruding shape T2-6 is formed substantially at the central portion of the second diffraction element 20-1.

The first diffraction element 10-1 has a deflection function of deflecting incident light L0-6 to the entry side of the incident light L0-6 on the first diffraction element 10, the incident light L0-6 being projected with a predetermined wavelength at a predetermined incident angle to the first diffraction element 10-1. The first diffraction element 10-1 emits first diffracted light L1-6 deflected by the deflection function.

As illustrated in FIG. 26, in the three-dimensional coordinate system of the light guide device 6, the normal line of the first diffraction element 10-1 extends from the origin point of the three-dimensional coordinate system in +z axis direction (upward direction in FIG. 26), the top surface (the upper surface in FIG. 26) of the first diffraction element 10-1 is an x-y plane (a plane extending from the near side to the remote side in the drawing) (if the upward-protruding shape T1-6 is formed at a low protrusion level, the x-y plane can approximate to the top surface of the first diffraction element 10 as follows, whereas if the upward-protruding shape T1-6 is formed at a high protrusion level, the x-y plane comes into contact with the origin point of the three-dimensional coordinate system in the region of the top surface of the first diffraction element 10-1). The incident light L0-6 on the first diffraction element 10-1 is light directed to the origin point of the three-dimensional coordinate system from any point in +z axis direction (upward direction in FIG. 26), in the range of the second quadrant of an x-y coordinate system (a coordinate system directed from the near side to the remote side in the drawing), and in the range of the third quadrant or on −x axis (a leftward axis in the drawing). The first diffracted light L1-6 emitted from the first diffraction element 10-1 is light directed from the origin point of the three-dimensional coordinate system to any point in +z axis direction (upward direction in FIG. 26), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on −y axis (an axis from the remote side to the near side in the drawing). The light is deflected in the regular reflection direction with respect to the incident light L0-6.

The second diffraction element 20-1 has a lens function for the first diffracted light L1-6 that is projected with a predetermined wavelength at a predetermined incident angle to the second diffraction element 20-1.

The second diffraction element 20-1 has a deflection function of deflecting the first diffracted light L1-6 to the entry side of the first diffracted light L1-6 on the second diffraction element 20-1, the first diffracted light L1-6 being projected with a predetermined wavelength at a predetermined incident angle to the second diffraction element 20-1. The second diffraction element 20-1 emits second diffracted light L2-6 deflected by the deflection function.

In the light guide device 6, the direction of the incident angle of the incident light L0-6 and the direction of the diffraction angle of the first diffracted light L1-6 are different from the optical axis direction of the second diffraction element 20-1.

The contents of the light guide device according to the sixth embodiment of the present technique can be applied to the light guide devices according to the first to fifth embodiments of the present technique and after-mentioned light guide devices according to seventh to eleventh embodiments of the present technique unless any technical contradictions arise.

### 9. Light Guide Device of Seventh Embodiment of Present Technique

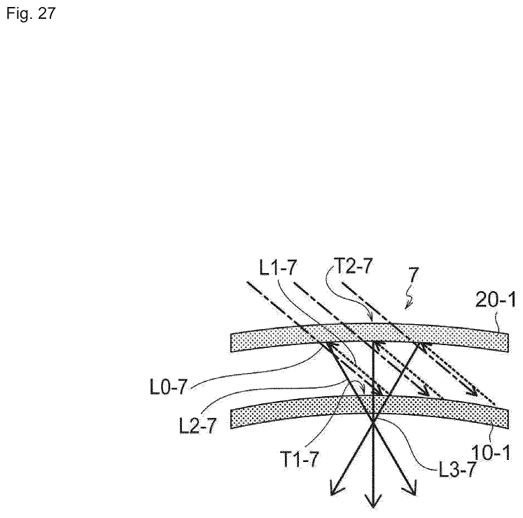

Referring to FIG. 27, a light guide device according to a seventh embodiment of the present technique will be described below.

FIG. 27 illustrates a configuration example of the light guide device according to the seventh embodiment of the present technique. Specifically, FIG. 27 illustrates a light guide device 7.

### (Light Guide Device 7)

The light guide device 7 includes at least an optical element configured with a first diffraction element 10-1 and a second diffraction element 20-1 that are opposed to each other. In the light guide device 7, an image is formed on the basis of image display light (display light) L3-7 that passes through the first diffraction element 10-1 and is emitted out of the light guide device 7. In the light guide device 7, the first diffraction element 10-1 and the second diffraction element 20-1 may be disposed close to each other. The first diffraction element 10-1 and the second diffraction element 20-1 may be disposed in contact, which is not illustrated in FIG. 27.

In the light guide device 7, the first diffraction element 10-1 has a curved shape such that an upward-protruding shape T1-7 is formed substantially at the central portion of the first diffraction element 10-1, and the second diffraction element 20-1 has a curved shape such that an upward-protruding shape T2-7 is formed substantially at the central portion of the second diffraction element 20-1.

The first diffraction element 10-1 has a deflection function of deflecting incident light L0-7 to the entry side of the incident light L0-7 on the first diffraction element 10, the incident light L0-7 being projected with a predetermined wavelength at a predetermined incident angle to the first diffraction element 10-1. The first diffraction element 10-1 emits first diffracted light L1-7 deflected by the deflection function.

As illustrated in FIG. 27, in the three-dimensional coordinate system of the light guide device 7, the normal line of the first diffraction element 10-1 extends from the origin point of the three-dimensional coordinate system in +z axis direction (upward direction in FIG. 27), the top surface (the upper surface in FIG. 27) of the first diffraction element 10-1 is an x-y plane (a plane extending from the near side to the remote side in the drawing) (if the upward-protruding shape T1-7 is formed at a low protrusion level, the x-y plane can approximate to the top surface of the first diffraction element 10-1 as follows, whereas if the upward-protruding shape T1-7 is formed at a high protrusion level, the x-y plane comes into contact with the origin point of the three-dimensional coordinate system in the region of the top surface of the first diffraction element 10-1). The incident light L0-7 on the first diffraction element 10-1 is light directed to the origin point of the three-dimensional coordinate system from any point in +z axis direction (upward direction in FIG. 27), in the range of the second quadrant of an x-y coordinate system (a coordinate system directed from the near side to the remote side in the drawing), and in the range of the third quadrant or on −x axis (leftward axis in the drawing). The first diffracted light L1-7 emitted from the first diffraction element 10-1 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (leftward axis in the drawing). The light is deflected in the incident direction with respect to the incident light L0-7.

The second diffraction element 20-1 has a lens function for the first diffracted light L1-7 that is projected with a predetermined wavelength at a predetermined incident angle to the second diffraction element 20-1.

The second diffraction element 20-1 has a deflection function of deflecting the first diffracted light L1-7 to the entry side of the first diffracted light L1-7 on the second diffraction element 20-1, the first diffracted light L1-7 being projected with a predetermined wavelength at a predetermined incident angle to the second diffraction element 20-1. The second diffraction element 20-1 emits second diffracted light L2-7 deflected by the deflection function.

In the light guide device 7, the direction of the incident angle of the incident light L0-7 and the direction of the diffraction angle of the first diffracted light L1-7 are different from the optical axis direction of the second diffraction element 20-1.

The contents of the light guide device according to the seventh embodiment of the present technique can be applied to the light guide devices according to the first to sixth embodiments of the present technique and after-mentioned light guide devices according to eighth to eleventh embodiments of the present technique unless any technical contradictions arise.

### 10. Light Guide Device of Eighth Embodiment of Present Technique

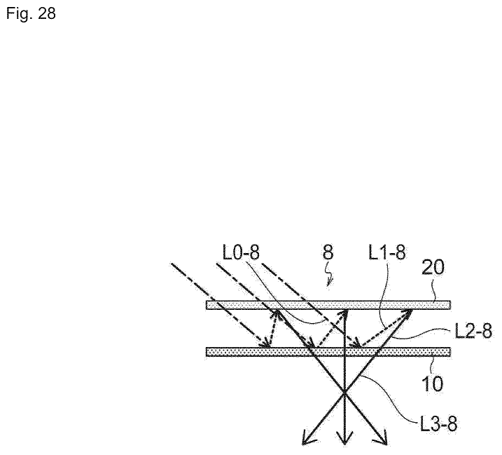

Referring to FIG. 28, a light guide device according to an eighth embodiment of the present technique will be described below. In the light guide device according to the eighth embodiment of the present technique, a first diffraction element is an HOE that does not merely deflect light but also diffracts light in a substantially regular reflection direction with any optical aberration (a dispersing or condensing effect or a wavefront correcting effect), and a second diffraction element is a reflection lens HOE that condenses or disperses an incident light beam (diffracted light beam) from the first diffraction element.

FIG. 28 illustrates a configuration example of the light guide device according to the eighth embodiment of the present technique. Specifically, FIG. 28 illustrates a light guide device 8.

### (Light Guide Device 8)

The light guide device 8 includes at least an optical element configured with a first diffraction element 10 and a second diffraction element 20 that are opposed to each other. In the light guide device 8, an image is formed on the basis of image display light (display light) L3-8 that passes through the first diffraction element 10 and is emitted out of the light guide device 8. In the light guide device 8, the first diffraction element 10 and the second diffraction element 20 may be disposed close to each other. The first diffraction element 10 and the second diffraction element 20 may be disposed in contact, which is not illustrated in FIG. 28.

The first diffraction element 10 has a deflection function of deflecting incident light L0-8 to the entry side of the incident light L0-8 on the first diffraction element 10, the incident light L0-8 being projected with a predetermined wavelength at a predetermined incident angle to the first diffraction element 10. The first diffraction element 10 emits first diffracted light L1-8 deflected by the deflection function.

As illustrated in FIG. 28, in the three-dimensional coordinate system of the light guide device 8, the normal line of the first diffraction element 10 extends from the origin point of the three-dimensional coordinate system in +z axis direction (upward direction in FIG. 28), the top surface (the upper surface in FIG. 28) of the first diffraction element 10 is an x-y plane (a plane extending from the near side to the remote side in the drawing), the incident light L0-8 on the first diffraction element 10 is light directed to the origin point of the three-dimensional coordinate system from any point in +z axis direction (upward direction in FIG. 28), in the range of the second quadrant of an x-y coordinate system (a coordinate system directed from the near side to the remote side in the drawing), and in the range of the third quadrant or on −x axis (a leftward axis in the drawing). The first diffracted light L1-8 emitted from the first diffraction element 10 is light directed from the origin point of the three-dimensional coordinate system to any point in +z axis direction (upper direction in FIG. 28), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on y axis (an axis from the remote side to the near side in the drawing). The light is deflected in the regular reflection direction with respect to the incident light L0-8.

The second diffraction element 20 has a lens function for the first diffracted light L1-8 that is projected with a predetermined wavelength at a predetermined incident angle to the second diffraction element 20.

The second diffraction element 20 has a deflection function of deflecting the first diffracted light L1-8 to the entry side of the first diffracted light L1-8 on the second diffraction element 20, the first diffracted light L1-8 being projected with a predetermined wavelength at a predetermined incident angle to the second diffraction element 20. The second diffraction element 20 emits second diffracted light L2-8 deflected by the deflection function.

In the light guide device 8, the direction of the incident angle of the incident light L0-8 and the direction of the diffraction angle of the first diffracted light L1-8 are different from the optical axis direction of the second diffraction element 20.

The contents of the light guide device according to the eighth embodiment of the present technique can be applied to the light guide devices according to the first to seventh embodiments of the present technique and after-mentioned light guide devices according to ninth to eleventh embodiments of the present technique unless any technical contradictions arise.

### 11. Light Guide Device of Ninth Embodiment of Present Technique

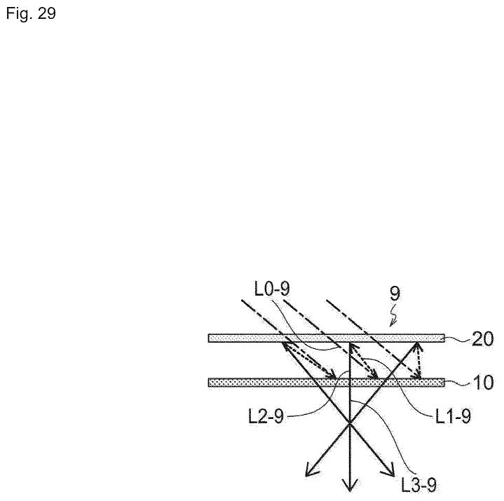

Referring to FIG. 29, a light guide device according to a ninth embodiment of the present technique will be described below. In the light guide device according to the ninth embodiment of the present technique, a first diffraction element is an HOE that does not merely deflect light but also diffracts light in an incident direction or a direction different from the incident direction and a regular reflection direction with any optical aberration (a dispersing or condensing effect or a wavefront correcting effect), and a second diffraction element is a reflection lens HOE that condenses or disperses an incident light beam (diffracted light beam) from the first diffraction element.

FIG. 29 illustrates a configuration example of the light guide device according to the ninth embodiment of the present technique. Specifically, FIG. 29 illustrates a light guide device 9.

### (Light Guide Device 9)

The light guide device 9 includes at least an optical element configured with a first diffraction element 10 and a second diffraction element 20 that are opposed to each other. In the light guide device 9, an image is formed on the basis of image display light (display light) L3-9 that passes through the first diffraction element 10 and is emitted out of the light guide device 9. In the light guide device 9, the first diffraction element 10 and the second diffraction element 20 may be disposed close to each other. The first diffraction element 10 and the second diffraction element 20 may be disposed in contact, which is not illustrated in FIG. 29.

The first diffraction element 10 has a deflection function of deflecting incident light L0-9 to the entry side of the incident light L0-9 on the first diffraction element 10, the incident light L0-9 being projected with a predetermined wavelength at a predetermined incident angle to the first diffraction element 10. The first diffraction element 10 emits first diffracted light L1-9 deflected by the deflection function.

As illustrated in FIG. 29, in the three-dimensional coordinate system of the light guide device 9, the normal line of the first diffraction element 10 extends from the origin point of the three-dimensional coordinate system in +z axis direction (upward direction in FIG. 29), the top surface (the upper surface in FIG. 29) of the first diffraction element 10 is an x-y plane (a plane extending from the near side to the remote side in the drawing), the incident light L0-9 on the first diffraction element 10 is light directed to the origin point of the three-dimensional coordinate system from any point in +z axis direction (upward direction in FIG. 29), in the range of the second quadrant of an x-y coordinate system (a coordinate system directed from the near side to the remote side in the drawing), and in the range of the third quadrant or on −x axis (a leftward axis in the drawing). The first diffracted light L1-9 emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in a direction different from the regular reflection direction and the incident direction with respect to the incident light L0-9.

The second diffraction element 20 has a lens function for the first diffracted light L1-9 that is projected with a predetermined wavelength at a predetermined incident angle to the second diffraction element 20.

The second diffraction element 20 has a deflection function of deflecting the first diffracted light L1-9 to the entry side of the first diffracted light L1-9 on the second diffraction element 20, the first diffracted light L1-9 being projected with a predetermined wavelength at a predetermined incident angle to the second diffraction element 20. The second diffraction element 20 emits second diffracted light L2-9 deflected by the deflection function.

In the light guide device 9, the direction of the incident angle of the incident light L0-9 and the direction of the diffraction angle of the first diffracted light L1-9 are different from the optical axis direction of the second diffraction element 20.

The contents of the light guide device according to the ninth embodiment of the present technique can be applied to the light guide devices according to the first to eighth embodiments of the present technique and after-mentioned light guide devices according to tenth and eleventh embodiments of the present technique unless any technical contradictions arise.

### 12. Light Guide Device of Tenth Embodiment of Present Technique

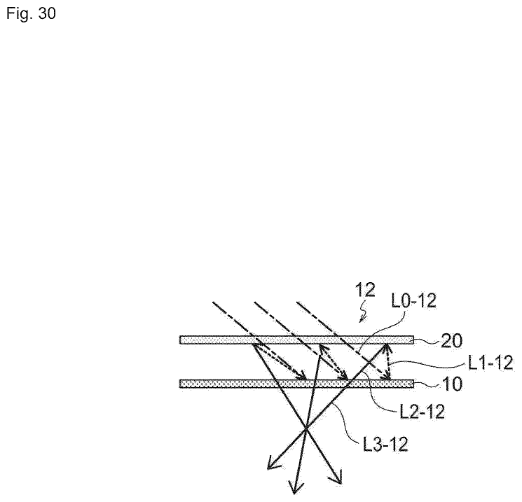

Referring to FIG. 30, a light guide device according to a tenth embodiment of the present technique will be described below. A feature of the light guide device according to the tenth embodiment of the present technique is that a light beam entering a first diffraction element forms an angle different from the angle of a light beam entering a second diffraction element and the angle of a light beam diffracted by the second diffraction element. Another feature is that the lens optical axis of the second diffraction element tilts from a vertical line with respect to the second diffraction element. Thus, in the case of a configuration where a light beam passes though the second diffraction element and enters the first diffraction element, the light guide device does not have the effect of condensing (convex lens) or dispersing (concave lens) a light beam incident on the second diffraction element and reflecting and diffracting the light beam. This allows the passage of the light beam incident on the second diffraction element.

FIG. 30 illustrates a configuration example of the light guide device according to the tenth embodiment of the present technique. Specifically, FIG. 30 illustrates a light guide device 12.

### (Light Guide Device 12)

The light guide device 12 includes at least an optical element configured with a first diffraction element 10 and a second diffraction element 20 that are opposed to each other. In the light guide device 12, an image is formed on the basis of image display light (display light) L3-12 that passes through the first diffraction element 10 and is emitted out of the light guide device 12. In the light guide device 12, the first diffraction element 10 and the second diffraction element 20 may be disposed close to each other. The first diffraction element 10 and the second diffraction element 20 may be disposed in contact, which is not illustrated in FIG. 30.

The first diffraction element 10 has a deflection function of deflecting incident light L0-12 to the entry side of the incident light L0-12 on the first diffraction element 10, the incident light L0-12 being projected with a predetermined wavelength at a predetermined incident angle to the first diffraction element 10. The first diffraction element 10 emits first diffracted light L1-12 deflected by the deflection function.

As illustrated in FIG. 30, in the three-dimensional coordinate system of the light guide device 12, the normal line of the first diffraction element 10 extends from the origin point of the three-dimensional coordinate system in +z axis direction (upward direction in FIG. 30), the top surface (the upper surface in FIG. 30) of the first diffraction element 10 is an x-y plane (a plane extending from the near side to the remote side in the drawing), the incident light L0-12 on the first diffraction element 10 is light directed to the origin point of the three-dimensional coordinate system from any point in +z axis direction (upward direction in FIG. 30), in the range of the second quadrant of an x-y coordinate system (a coordinate system directed from the near side to the remote side in the drawing), and in the range of the third quadrant or on −x axis (a leftward axis in the drawing). The first diffracted light L1-12 emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in a direction different from the regular reflection direction and the incident direction with respect to the incident light L0-12.

The second diffraction element 20 has a lens function for the first diffracted light L1-12 that is projected with a predetermined wavelength at a predetermined incident angle to the second diffraction element 20.

The second diffraction element 20 has a deflection function of deflecting the first diffracted light L1-12 to the entry side of the first diffracted light L1-12 on the second diffraction element 20, the first diffracted light L1-12 being projected with a predetermined wavelength at a predetermined incident angle to the second diffraction element 20. The second diffraction element 20 emits second diffracted light L2-12 deflected by the deflection function.

In the light guide device 12, the direction of the incident angle of the incident light L0-12 and the direction of the diffraction angle of the first diffracted light L1-12 are different from the optical axis direction of the second diffraction element 20.

The contents of the light guide device according to the tenth embodiment of the present technique can be applied to the light guide devices according to the first to ninth embodiments of the present technique and an after-mentioned light guide device according to an eleventh embodiment of the present technique unless any technical contradictions arise.

### 13. Light Guide Device of Eleventh Embodiment of Present Technique

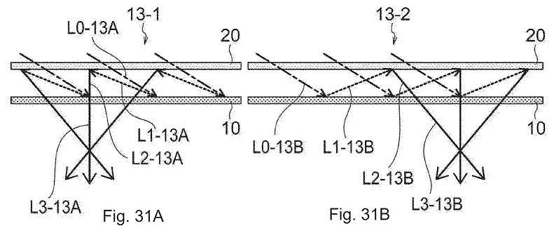

Referring to FIG. 31, a light guide device according to an eleventh embodiment of the present technique will be described below. A feature of the light guide device according to the eleventh embodiment of the present technique is that the angle of a projected and diffracted light beam of a first diffraction element is larger than the light-beam traveling angle of the lens function of a second diffraction element (an incident angle from the first diffraction element to the second diffraction element is larger than FOV/2). A region where diffraction efficiency decreases is excluded from an FOV.

FIG. 31 illustrates a configuration example of the light guide device according to the eleventh embodiment of the present technique. Specifically, FIG. 31A illustrates a light guide device 13-1, and FIG. 31B illustrates a light guide device 13-2.

### (Light Guide Device 13-1)

The light guide device 13-1 includes at least an optical element configured with a first diffraction element 10 and a second diffraction element 20 that are opposed to each other. In the light guide device 13-1, an image is formed on the basis of image display light (display light) L3-13A that passes through the first diffraction element 10 and is emitted out of the light guide device 13-1. In the light guide device 13-1, the first diffraction element 10 and the second diffraction element 20 may be disposed close to each other. The first diffraction element 10 and the second diffraction element 20 may be disposed in contact, which is not illustrated in FIG. 31A.

The first diffraction element 10 has a deflection function of deflecting incident light L0-13A to the entry side of the incident light L0-13A on the first diffraction element 10, the incident light L0-13A being projected with a predetermined wavelength at a predetermined incident angle to the first diffraction element 10. The first diffraction element 10 emits first diffracted light L1-13A deflected by the deflection function.

As illustrated in FIG. 31A, in the three-dimensional coordinate system of the light guide device 13-1, the normal line of the first diffraction element 10 extends from the origin point of the three-dimensional coordinate system in +z axis direction (upward direction in FIG. 31A), the top surface (the upper surface in FIG. 31A) of the first diffraction element 10 is an x-y plane (a plane extending from the near side to the remote side in the drawing), the incident light L0-13A on the first diffraction element 10 is light directed to the origin point of the three-dimensional coordinate system from any point in +z axis direction (upward direction in FIG. 31A), in the range of the second quadrant of an x-y coordinate system (a coordinate system directed from the near side to the remote side in the drawing), and in the range of the third quadrant or on −x axis (a leftward axis in the drawing). The first diffracted light L1-13A emitted from the first diffraction element 10 is light directed from the origin point of the three-dimensional coordinate system to any point in +z axis direction, in the range of the second quadrant of the x-y coordinate system (a coordinate system directed from the near side to the remote side in the drawing), in the range of the third quadrant or on −x axis (a leftward axis in the drawing). The light is deflected in a direction different from the regular reflection direction and the incident direction with respect to the incident light L0-13A.

The second diffraction element 20 has a lens function for the first diffracted light L1-13A that is projected with a predetermined wavelength at a predetermined incident angle to the second diffraction element 20.

The second diffraction element 20 has a deflection function of deflecting the first diffracted light L1-13A to the entry side of the first diffracted light L1-13A on the second diffraction element 20, the first diffracted light L1-13A being projected with a predetermined wavelength at a predetermined incident angle to the second diffraction element 20. The second diffraction element 20 emits second diffracted light L2-13A deflected by the deflection function.

In the light guide device 13-1, the direction of the incident angle of the incident light L0-13A and the direction of the diffraction angle of the first diffracted light L1-13A are different from the optical axis direction of the second diffraction element 20.

### (Light Guide Device 13-2)

The light guide device 13-2 includes at least an optical element configured with a first diffraction element 10 and a second diffraction element 20 that are opposed to each other. In the light guide device 13-2, an image is formed on the basis of image display light (display light) L3-13B that passes through the first diffraction element 10 and is emitted out of the light guide device 13-2. In the light guide device 13-2, the first diffraction element 10 and the second diffraction element 20 may be disposed close to each other. The first diffraction element 10 and the second diffraction element 20 may be disposed in contact, which is not illustrated in FIG. 31B.

The first diffraction element 10 has a deflection function of deflecting incident light L0-13B to the entry side of the incident light L0-13B on the first diffraction element 10, the incident light L0-13B being projected with a predetermined wavelength at a predetermined incident angle to the first diffraction element 10. The first diffraction element 10 emits first diffracted light L1-13B deflected by the deflection function.

As illustrated in FIG. 31B, in the three-dimensional coordinate system of the light guide device 13-2, the normal line of the first diffraction element 10 extends from the origin point of the three-dimensional coordinate system in +z axis direction (upward direction in FIG. 31B), the top surface (the upper surface in FIG. 31B) of the first diffraction element 10 is an x-y plane (a plane extending from the near side to the remote side in the drawing), the incident light L0-13B on the first diffraction element 10 is light directed to the origin point of the three-dimensional coordinate system from any point in +z axis direction (upward direction in FIG. 31B), in the range of the second quadrant of an x-y coordinate system (a coordinate system directed from the near side to the remote side in the drawing), and in the range of the third quadrant or on −x axis (a leftward axis in the drawing). The first diffracted light L1-13B emitted from the first diffraction element 10 is light directed from the origin point of the three-dimensional coordinate system to any point in +z axis direction (upper direction in FIG. 31B), in the range of the first quadrant of the x-y coordinate system (a coordinate system directed from the near side to the remote side in the drawing), in the range of the fourth quadrant, on +x axis (a rightward axis in the drawing), on +y axis (an axis from the near side to the remote side in the drawing), or on −y axis (an axis from the remote side to the near side in the drawing). The light is deflected in the regular reflection direction with respect to the incident light L0-13B.

The second diffraction element 20 has a lens function for the first diffracted light L1-13B that is projected with a predetermined wavelength at a predetermined incident angle to the second diffraction element 20.

The second diffraction element 20 has a deflection function of deflecting the first diffracted light L1-13B to the entry side of the first diffracted light L1-13B on the second diffraction element 20, the first diffracted light L1-13B being projected with a predetermined wavelength at a predetermined incident angle to the second diffraction element 20. The second diffraction element 20 emits second diffracted light L2-13B deflected by the deflection function.

In the light guide device 13-2, the direction of the incident angle of the incident light L0-13B and the direction of the diffraction angle of the first diffracted light L1-13B are different from the optical axis direction of the second diffraction element 20.

The contents of the light guide device according to the eleventh embodiment of the present technique can be applied to the light guide devices according to the first to tenth embodiments of the present technique unless any technical contradictions arise.

### 14. Display Device of Twelfth Embodiment of Present Technique

A display device according to a twelfth embodiment of the present technique is a display device including a frame to be mounted the head of a user, a light source device (e.g., an image projection device) mounted on the frame, and a light guide device including a light guide system having an optical element attached to the surface of an eyeball or embedded in an eyeball, wherein light is emitted from the light source device to the light guide device to display an image on a retina.

The light guide device may be any one of the light guide devices according to the first to eleventh embodiments of the present technique.

For example, the light source device may include a display element with a plurality of pixels placed in a two-dimensional array. The display element may have, for example, a light source array in which light sources (pixels) such as OLEDs (Organic light emitting diodes) are placed in an array, or may have a light source and image forming elements (a liquid crystal panel, a digital mirror device, a scan mirror) that form an image using light from the light source.

The light source device may have an optical system including a lens and a mirror in addition to the display element.

### 15. Display System of Thirteenth Embodiment of Present Technique

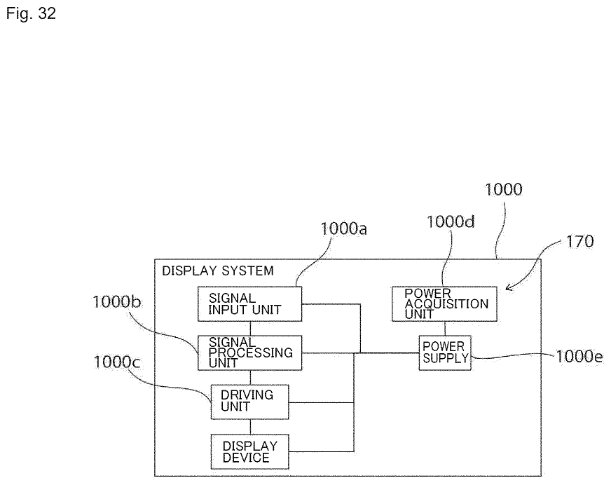

Referring to FIG. 32, a display system 1000 according to a fifteenth embodiment of the present technique will be described below. FIG. 32 is a block diagram illustrating the functions of the display system 1000. The display system 1000 includes a display device (e.g., the display device according to the twelfth embodiment) and a controller 170. The controller 170 includes, for example, a signal input unit 1000a, a signal processing unit 1000b, a driving unit 1000c, a power acquisition unit 1000d, and a power supply 1000e.

The signal input unit 1000a receives a video signal from a video signal output device (e.g., a smartphone, a personal computer, memory, or an imaging device).

The signal processing unit 1000b processes a video signal inputted through the signal input unit 1000a and generates a driving signal (modulating signal) for driving the display device.

The driving unit 1000c drives the display device by applying the driving signal from the signal processing unit 1000b to the display device.

The power acquisition unit 1000d acquires power from the power supply 1000e via wired or wireless connection and distributes the power to the signal input unit 1000a, the signal processing unit 1000b, the driving unit 1000c, and the display device.

The power supply 1000e may be a storage battery (e.g., a battery or a secondary battery) or a power generation source (a fuel cell, electromagnetic induction, or energy harvest or the like).

Note that embodiments according to the present technique are not limited to the foregoing embodiments and can be changed in various ways without departing from the gist of the present technique. For example, at least one of the first and second diffraction elements may be a DOE (Diffractive Optical Element) or a meta-material optical element. The present technique is applicable to, for example, a DOE and a meta-material optical element as well as an HOE. The optical element including the first and second diffraction elements may include at least one diffraction element in addition to the first and second diffraction elements.

Furthermore, the effects described in the present specification are merely exemplary and not intended to be limited, and other effects may be provided as well.

The present technique can also be configured as follows:
(1) A light guide device including a light guide system that guides light emitted from a light source device to eyeballs,
wherein the light guide system has an optical element including first and second diffraction elements that are opposed to each other, light emitted from the light source device and projected to the first diffraction element at a predetermined incident angle is reflected and diffracted at the first diffraction element, the light transmitted through the first diffraction element is reflected and diffracted at the second diffraction element, and the light transmitted through the first diffraction element via the second diffraction element is guided to the eyeballs,
the second diffraction element has a lens function, and
the first and second diffraction elements vary in refractive index difference and/or thickness.
(2) The light guide device according to claim 1, wherein the first and second diffraction elements vary in the product of a refractive index difference and a thickness.
(3) The light guide device according to (1) or (2), wherein a first product as the product of the refractive index difference and the thickness of the first diffraction element is larger than a second product as the product of the refractive index difference and the thickness of the second diffraction element.
(4) The light guide device according to any one of (1) to (3), wherein the first and second diffraction elements have different refractive index differences.
(5) The light guide device according to any one of (1) to (4), wherein the first diffraction element has a larger refractive index difference than the second diffraction element.
(6) The light guide device according to any one of (1) to (5), wherein the first and second diffraction elements have different thicknesses.
(7) The light guide device according to any one of (1) to (6), wherein the second diffraction element has a larger thickness than the first diffraction element.
(8) The light guide device according to any one of (1) to (7), wherein the first and second diffraction elements vary in refractive index difference and thickness.
(9) The light guide device according to any one of (1) to (8), wherein the first diffraction element has a larger refractive index difference than the second diffraction element, and the second diffraction element has a larger thickness than the first diffraction element.
(10) The light guide device according to any one of (1) to (9), wherein the first and second diffraction elements are identical in thickness, and the first diffraction element has a larger refractive index difference than the second diffraction element.
(11) The light guide device according to any one of (1) to (10), wherein the first and second diffraction elements are identical in refractive index difference, and the second diffraction element has a larger thickness than the first diffraction element.
(12) The light guide device according to any one of (1) to (11), wherein when the first and second diffraction elements have thicknesses in μm, the first product is 0.2 μm or more, and the first diffraction element has a thickness of 1 μm to 100 μm.
(13) The light guide device according to any one of (1) to (12), wherein when the first and second diffraction elements have thicknesses in μm, the first product is 0.4 μm or more, and the first diffraction element has a thickness of 2 μm to 30 μm.
(14) The light guide device according to any one of (1) to (13), wherein when the first and second diffraction elements have thicknesses in μm, the second product is 0.1 μm or less, and the second diffraction element has a thickness of 1 μm to 100 μm.
(15) The light guide device according to any one of (1) to (14), wherein when the first and second diffraction elements have thicknesses in μm, the second product is 0.02 μm or less, and the second diffraction element has a thickness of 10 μm to 40 μm.
(16) The light guide device according to any one of (1) to (15), wherein the optical axis of the second diffraction element and the normal line of the first diffraction element are parallel to each other.
(17) The light guide device according to any one of (1) to (15), wherein the optical axis of the second diffraction element and the normal line of the first diffraction element are non-parallel.
(18) The light guide device according to any one of (1) to (17), wherein light projected to the first diffraction element at the predetermine incident angle is light that is emitted from the light source device and is transmitted through the second diffraction element.
(19) The light guide device according to any one of (1) to (18), wherein the light guide system further includes a light guide plate that is at least partially disposed between the first and second diffraction elements and guides light from the light source device, and light projected to the first diffraction element at the predetermine incident angle is light that is emitted from the light source device and is guided by the light guide plate.
(20) The light guide device according to any one of (1) to (19), wherein the first diffraction element reflects and diffracts, in a regular reflection direction, light projected to the first diffraction element at the predetermine incident angle.
(21) The light guide device according to any one of (1) to (19), wherein the first diffraction element reflects and diffracts, in an incident direction, light projected to the first diffraction element at the predetermine incident angle.
(22) The light guide device according to any one of (1) to (19), wherein the first diffraction element reflects and diffracts light projected to the first diffraction element at the predetermine incident angle, in a direction different from both of the regular reflection direction and the incident direction.
(23) The light guide device according to any one of (1) to (22), wherein at least one of the first and second diffraction elements has a curved element surface.
(24) The light guide device according to any one of (1) to (23), wherein at least one of the first and second diffraction elements is a hologram optical element.
(25) The light guide device according to any one of (1) to (24), wherein the optical element is attached to or embedded into the eyeball.
(26) A display device including: the light guide device according to any one of (1) to (25); and
the light source device.
(27) The display device according to (26), wherein the light source device and the light guide device are integrally provided.
(28) The display device according to (26) or (27), wherein the display device is a head-mounted display.
(29) A display system including: the display device according to any one of (26) to (28); and
a controller that controls the display device.

### REFERENCE SIGNS LIST

1(1-1, 1-2), 2, 3(3-1, 3-2), 4(4-1, 4-2), 5, 6, 7, 8, 9, 10, 11, 12, 13(13-1, 13-2) Light guide device
10, 10-1 First diffraction element
20, 20-1 Second diffraction element
500 Light guide plate
1000 Display system

1. A light guide device comprising a light guide system that guides light emitted from a light source device to eyeballs,
wherein the light guide system has an optical element including first and second diffraction elements that are opposed to each other, light emitted from the light source device and projected to the first diffraction element at a predetermined incident angle is reflected and diffracted at the first diffraction element, the light transmitted through the first diffraction element is reflected and diffracted at the second diffraction element, and the light transmitted through the first diffraction element via the second diffraction element is guided to the eyeballs,
the second diffraction element has a lens function, and
the first and second diffraction elements vary in refractive index difference and/or thickness.

2. The light guide device according to claim 1, wherein the first and second diffraction elements vary in a product of a refractive index difference and a thickness.

3. The light guide device according to claim 1, wherein a first product as a product of a refractive index difference and a thickness of the first diffraction element is larger than a second product as a product of a refractive index difference and a thickness of the second diffraction element.

4. The light guide device according to claim 1, wherein the first and second diffraction elements have different refractive index differences.

5. The light guide device according to claim 1, wherein the first diffraction element has a larger refractive index difference than the second diffraction element.

6. The light guide device according to claim 1, wherein the first and second diffraction elements have different thicknesses.

7. The light guide device according to claim 1, wherein the second diffraction element has a larger thickness than the first diffraction element.

8. The light guide device according to claim 1, wherein the first and second diffraction elements vary in refractive index difference and thickness.

9. The light guide device according to claim 1, wherein the first diffraction element has a larger refractive index difference than the second diffraction element, and the second diffraction element has a larger thickness than the first diffraction element.

10. The light guide device according to claim 1, wherein the first and second diffraction elements are identical in thickness, and the first diffraction element has a larger refractive index difference than the second diffraction element.

11. The light guide device according to claim 1, wherein the first and second diffraction elements are identical in refractive index difference, and the second diffraction element has a larger thickness than the first diffraction element.

12. The light guide device according to claim 3, wherein when the first and second diffraction elements have thicknesses in μm, the first product is 0.2 or more, and the first diffraction element has a thickness of 1 μm to 100 μm.

13. The light guide device according to claim 3, wherein when the first and second diffraction elements have thicknesses in μm, the first product is 0.4 or more, and the first diffraction element has a thickness of 2 μm to 30 μm.

14. The light guide device according to claim 3, wherein when the first and second diffraction elements have thicknesses in μm, the second product is 0.1 or less, and the second diffraction element has a thickness of 1 μm to 100 μm.

15. The light guide device according to claim 3, wherein when the first and second diffraction elements have thicknesses in μm, the second product is 0.02 or less, and the second diffraction element has a thickness of 10 μm to 40 μm.

16. The light guide device according to claim 1, wherein an optical axis of the second diffraction element and a normal line of the first diffraction element are parallel to each other.

17. The light guide device according to claim 1, wherein an optical axis of the second diffraction element and a normal line of the first diffraction element are non-parallel.

18. The light guide device according to claim 1, wherein light projected to the first diffraction element at the predetermine incident angle is light that is emitted from the light source device and is transmitted through the second diffraction element.

19. The light guide device according to claim 1, wherein the light guide system further includes a light guide plate that is at least partially disposed between the first and second diffraction elements and guides light from the light source device, and
light projected to the first diffraction element at the predetermine incident angle is light that is emitted from the light source device and is guided by the light guide plate.

20. The light guide device according to claim 1, wherein the first diffraction element reflects and diffracts, in a regular reflection direction, light projected to the first diffraction element at the predetermine incident angle.

21. The light guide device according to claim 1, wherein the first diffraction element reflects and diffracts, in an incident direction, light projected to the first diffraction element at the predetermine incident angle.

22. The light guide device according to claim 1, wherein the first diffraction element reflects and diffracts light projected to the first diffraction element at the predetermine incident angle, in a direction different from both of a regular reflection direction and an incident direction.

23. The light guide device according to claim 1, wherein at least one of the first and second diffraction elements has a curved element surface.

24. The light guide device according to claim 1, wherein at least one of the first and second diffraction elements is a hologram optical element.

25. The light guide device according to claim 1, wherein the optical element is attached to or embedded into the eyeball.

26. A display device comprising: the light guide device according to claim 1; and
the light source device.

27. The display device according to claim 26, wherein the light source device and the light guide device are integrally provided.

28. The display device according to claim 26, wherein the display device is a head-mounted display.

29. A display system comprising: the display device according to claim 26; and
a controller that controls the display device.
